SEQUENCE LISTING (10 sequences)

SEQ ID NO: 1 - DNA, 36 bp, Artificial Sequence
  synthetic primer
tttggatccgagatgaaaatggtaatatagcgaata

SEQ ID NO: 2 - DNA, 38 bp, Artificial Sequence
  synthetic primer
tttcccgggatacgttctcctttttgtataaaattaaa

SEQ ID NO: 3 - DNA, 38 bp, Artificial Sequence
  Synthetic primer
tttcccgggggtaattctaggtgatttttaaattatct

SEQ ID NO: 4 - DNA, 33 bp, Artificial Sequence
  Synthetic primer
tttgaattcatgtgccattgtttttaaaagttc

SEQ ID NO: 5 - DNA, 40 bp, Artificial Sequence
  Synthetic primer
tttggatcctttcattatgatagaaataacatagcagttg

SEQ ID NO: 6 - DNA, 65 bp, Artificial Sequence
  Synthetic primer
tttgaattcttattcacccgtagccggaagagcttgagctcctatctcatagccttttttagaaa

SEQ ID NO: 7 - DNA, 32 bp, Artificial Sequence
  Synthetic primer
tgtcgaggtaccttgagccttgatagtgcgag

SEQ ID NO: 8 - DNA, 32 bp, Artificial Sequence
  Synthetic primer
ctaacacggccgttaggggttagcctgtagat

SEQ ID NO: 9 - DNA, 4873 bp, Artificial sequence
  Bacillus anthracis cap operon - deletion/frameshift mutation in capD gene
ttgagccttgatagtgcgagaagacatatgaaaaacataaaaattgtaagaatattgaaacatgatgaggcaatacgcattgaacataggatttcagaattatactcagatgaattcggtgttgtatatgcagggaaccacctaatttttaattggtatcaacgactctacttaagtcgaaatatcttaataagcaagaaatcgaaaagcaggaagggattaatacagatgatcttcataataggtatatgtacagtgtttttgattatttatggtatatgggaacaacgttgccatcagaaaaggctcaattctatcccaattcgagtaaacataaatggaattcgaggtaaatctaccgttacaagactaattacaggtgttgtacaagaagcgaaatataagactgtagggaaaacaactggtacatctgcgcgaatgatatattggtttactgacgaggagcaaccgattaagcgccgtaaagaaggtcctaatatcggtgagcaacgcagggtagttaaagaggctgctgatttagaagcagaagcacttatttgtgaatgtatggcagttcaacccgattatcaaattatcttccaaaataaaatgattcaagcaaatgttggagtgattgtaaatgttttagaagatcatatggatgttatgggacctacacttgacgaagtagctgaagctttcactgctaccattccatataatggacatttagtcactattgaaagtgaatacttggattactttaaagaggttgcagaagagagaaatacaaaagtgattgttgcggataattctagaatttcagaagaattcttacgaaaatttgattacatggtcttcccagataatgcatcgcttgctttagcggtagcagaggctcttgggattgatgaggaaacagcattccgtggtatgttgaatgctcatccggatccaggagcaatgagaattacacgttttgctgaccaatctaagcctgcgttcttcgtaaatggttttgcagcgaatgatccctcatcaacattacgtatttgggaacgtgtggatgattttggatatagtaatctagctccaattgtaattatgaattgccgccctgaccgcgttgatcgtactgagcagtttgctagggatgttttgccatatattaaagcggaaatagttattgcgattggagaaacgactgcacctattacaagtgcttttgaaaaaggagatattccaacgcaagagtattggaacttagaaggctggtcaacaagtgaaattatgtctcgtatgcgtccatatttaaaaaatcggattgtatatggagtgggtaatattcatggtgcagctgagccattaatcgatatgattatggaagaacaaattggcaaaaagcaagcaaaagtgatttaagtggagggacaggaatgtttggatcagatttatatattgcattagtattaggagttacactgagccttatttttacagaaagaacaggtattttacctgcaggtttagttgtacctggttatttagcactcgtttttaatcagcccgtatttatgttggttgttttatttatcagtattttaacatatgtaatcgttacgtatggtgtttcaagattcatgattttatatggccgtagaaaatttgcggcaacgctaattacaggtatttgtttaaaacttttatttgattattgttatcctgttatgccatttgagatttttgaattccgtggtattggagttattgttccaggattaattgcaaatacaattcaaagacaagggttaccattaacaattggaactacaattttgttaagtggtgcaacatttgcaatcatgaatatttattacttattttaaggtgaggtagaatgagacgaaaattgacatttcaagaaaagttactgatctttattaagaaaaccaagaaaaaaaatcctcgttatgtagcaatcgtattacctcttatcgcagttatattaatagctgcgacatgggtacaacgtacagaagcagtagcaccagtaaaacatcgtgagaacgaaaaattgacgatgacgatggttggtgacattatgatgggacgtcacgtaaaagagattgttaatcgttacggtacagattatgtttttcgtcatgtttcgccatatttaaaaaactcagattacgtaagtgggaatttcgaacatcctgttttgttagaagataaaaagaattatcaaaaagcagataagaatattcacttaagtgcaaaagaagaaacagttaaggcagtaaaagaagccggatttacagtattaaatttggcgaataaccatatgacggattatggtgctaagggaactaaagatacaataaaggcctttaaagaagctgatcttgactatgtgggtgctggtgaaaatttcaaagatgtaaaaaatattgtgtatcaaaatgtaaatggtgttagggttgctactcttggatttacagatgcatttgtagcaggagctattgcaacgaaagaacaaccaggttcgttaagtatgaacccagatgtattacttaagcaaattagtaaggcaaaggatcctaaaaaaggtaatgctgatcttgtcgtagtaaatacgcactggggggaagaatacgataataaaccgagtcctagacaggaagccttagcaaaagcaatggttgatgcaggggcagatattattgtgggacaccatccgcatgtacttcaatcttttgatgtgtataagcaagggattatcttctatagtttaggtaactttgtgtttgaccaaggatggacaagaacaaaagatagtgcacttgtgcaatatcatttacgtgataatggtactgcaattcttgatgttgtacctttaaatattcaagagggatcaccaaaaccagttaccagtgcattggataaaaatcgtgtgtatcgtcaattaacaaaagatacatccaagggtgctctatggagtaaaaaagatgataaattggaaatcaaattaaatcataaacatgttattgaaaaaatgaaaaagagggaaaagcaagagcatcaagataagcaagaaaaagaaaatcaagtatcagtggagacaacaacttgaattcctttaaatggggaaagaagataattcttttctgtttgatagtcagcttaatggggggtatcggggtatcctgttctttcaataaaataaaagacagtgttaagcaaaaaattgatagtatgggtgataaaggaacttatggagtgagtgcctctcacccccttgcggttgaggaaggtatgaaagtattaaagaacggtggaagtgcagtagatgcagcgattgtggtctcatatgttttaggcgttgtagaactgcatgcctcaggaataggtgggggcggtggaatgctcattatatctaaagataaagaaacctttattgattatcgtgaaacaactccgtactttacaggaaaccaaaagccacatattggagtacccggatttgtggctggaatggagtatattcatgataattatggttcattaccgatgggtgagttattacaaccagccattaattatgcggaaaaagggttcaaggtagatgattccttaacaatgcgattagaccttgcgaagccacgtatttattctgataagctaagtatcttctatccgaatggtgaacctattgaaactggagaaacacttatccagacagatttagcgagaaccttaaagaagattcaaaaagaaggggctaaaggcttttatgaaggaggagtcgctagggcaatcagtaaaactgcaaaaatatcgttagaagatataaaaggatataaagtagaggtacgtaaaccagtaaaaggtaactacatgggatatgatgtttataccgctccaccacctttttcaggagttactttattacaaatgttgaaattagctgaaaagaaagaagtatataaagatgtagatcatacggcaacttatatgtctaaaatggaagagatttcaaggattgcctatcaagatagaaagaaaaacctaggggatccaattacgttaatatggatccaaataaaatggtgagtgacaaatatatatcaacaatgaagaatgagaatggtgatgcgctttcggaagcagagcatgaaagcacaacgcattttgttatcattgatagagatggaacggttgtctcttcaactaatacactaagcaatttctttggaacaggaaagtacacagcagggttcttcttaaataatcaattgcagaactttggaagtgagggatttaatagttatgaacctggtaaacgttcacgaacgtttatggcccccactgtattaaagaaagatggggaaacgatcggcattgggtcaccaggtggtaaccgtattccgcaaattttaaccccaatattggataaatatacgcatggtaagggtagcttgcaagacattatcaatgaataccgttttacttttgaaaaaaatacagcgtatacagagattcagctaagttcagaagtgaaaaatgagttatctagaaaaggattgaacgtaaagaagaaagtatcccctgccttttttggtggggtacaggccttaattaaagacgagagagataatgttatcaccggcgctggagatggcagaagaaatggaacttggaaatcaaataaataggaggtaatggagaaatggttaaaaaagtttttggatggattatgccgattttaattgtaggtttattacttgtaacaatggggacctttaaacgttcggaaacattaacgactgatgagcagaagaagattagtgattatctacaggctaacccctaa

SEQ ID NO: 10 - protein, 764 aa, Bacillus anthracis
MKKRKVLIPLMALSTILVSSTGNLEVIQAEVKQENRLLNESESSSQGLLGYYFSDLNFQAPMVVTSSTTGDLSIPSSELENIPSENQYFQSAIWSGFIKVKKSDEYTFATSADNHVTMWVDDQEVINKASNSNKIRLEKGRLYQIKIQYQRENPTEKGLDFKLYWTDSQNKKEVISSDNLQLPELKQKSSNSRKKRSTSAGPTVPDRDNDGIPDSLEVEGYTVDVKNKRTFLSPWISNIHEKKGLTKYKSSPEKWSTASDPYSDFEKVTGRIDKNVSPEARHPLVAAYPIVHVDMENIILSKNEDQSTQNTDSQTRTISKNTSTSRTHTSEVHGNAEVHASFFDIGGSVSAGFSNSNSSTVAIDHSLSLAGERTWAETMGLNTADTARLNANIRYVNTGTAPIYNVLPTTSLVLGKNQTLATIKAKENQLSQILAPNNYYPSKNLAPIALNAQDDFSSTPITMNYNQFLELEKTKQLRLDTDQVYGNIATYNFENGRVRVDTGSNWSEVLPQIQETTARIIFNGKDLNLVERRIAAVNPSDPLETTKPDMTLKEALKIAFGFNEPNGNLQYQGKDITEFDFNFDQQTSQNIKNQLAELNATNIYTVLDKIKLNAKMNILIRDKRFHYDRNNIAVGADESVVKEAHREVINSSTEGLLLNIDKDIRKILSGYIVEIEDTEGLKEVINDRYDMLNISSLRQDGKTFIDFKKYNDKLPLYISNPNYKVNVYAVTKENTIINPSENGDTSTNGIKKILIFSKKGYEIG

(19) United States
(12) Patent Application Publication
(10) Pub. No.: US 2014/0302084 A1
(43) Pub. Date: Oct. 9, 2014

(54) IMMUNOGENIC PROTEIN CONJUGATES AND METHOD FOR MAKING AND USING THE SAME

(71) Applicants: Olaf Schneewind, Chicago, IL (US); Dominique M. Missiakas, Chicago, IL (US); Yating Wang, Chicago, IL (US)

(72) Inventors: Olaf Schneewind, Chicago, IL (US); Dominique M. Missiakas, Chicago, IL (US); Yating Wang, Chicago, IL (US)

(73) Assignee: The University of Chicago, Chicago, IL (US)

(21) Appl. No.: 14/237,320

(22) PCT Filed: Aug. 5, 2012
(86) PCT No.: PCT/US2012/049673

§ 371 Date: May 27, 2014

Related U.S. Application Data
(60) Provisional application No. 61/515,733, filed on Aug. 5, 2011.

(51) Int. Cl. A61K 47/48; C12P 21/02

(52) U.S. Cl. CPC A61K 47/4833; C12P 21/02
USPC 424/190.1; 435/68.1

(57) ABSTRACT

Production of protein conjugate vaccines by use of transpeptidase enzymes, such as sortase enzymes. For example, homogenous immunoconjugates (e.g., a population of molecules having the same structure) formed by conjugating an antigenic polypeptide and a bacterial capsule component are provided. In certain aspects, methods for generating an immune response to B. anthracis by use of protective antigen-PDGA immunoconjugates are provided.

This application claims the benefit of priority to U.S. Provisional Patent Application Ser. No. 61/515,733, filed Aug. 5, 2011, hereby incorporated by reference in its entirety.

This invention was made with government support under 1-U54-AI-057153 and R01-AI069227 awarded by the National Institutes of Health. The government has certain rights in the invention.

### BACKGROUND OF THE INVENTION

A. Field of the Invention

Embodiments of this invention are directed generally to microbiology, medicine and immunology. In certain aspects, the invention is directed to immunoconjugate production and the treatment or prevention of Bacillus anthracis infection.

B. Background

The Gram-positive, spore forming bacterium Bacillus anthracis is the causative agent of anthrax, which is primarily a disease of herbivores. Following ingestion of infectious spores, B. anthracis germinate in host tissues and replicate as chains of vegetative bacilli, enclosed by a large poly-D-γ-glutamic acid (PDGA) capsule to prevent their clearance by phagocytes. Bacilli secrete three proteins—lethal factor (LF), edema factor (EF) and protective antigen (PA)—that assemble into the binary lethal (LF and PA) and edema (EF and PA) toxins. PA interacts with anthrax toxin receptors to translocate LF and EF into host cells, where toxins exert their zinc protease (LF) and adenylate cyclase (EF) functions. The two virulence strategies of B. anthracis, toxin secretion and capsule formation, are encoded by two large virulence plasmids pXO1 and pXO2. Loss of virulence plasmid occurs under laboratory conditions and prompted the development of attenuated vaccine strains, Pasteur (pXO1−, pXO2+) and Sterne (pXO1+, pXO2−).

Human infections with B. anthracis spores occurs following contact, ingestion or inhalation, giving rise to the cutaneous, gastrointestinal or respiratory forms of disease. Owing to ease of preparation and dissemination, B. anthracis spores have been used as biological weapons. To counter the catastrophic consequences of an anthrax aerosol attack, the United States stockpiles vaccine and its military personnel are immunized with anthrax vaccine adsorbed (AVA, BioThrax®) (15). AVA is the aluminum hydroxide adsorbed precipitate of B. anthracis V63340 77/-NP1—R (pXO1+, pXO2−) culture supernatants. PA is the principal immunogen of AVA and purified PA is being pursued as a next generation human anthrax vaccine. However, conventional B. anthracis vaccines require numerous boosters and a long period of administration. Thus, the deficiency in current B. anthracis vaccine compositions highlights the need for improved immunogens to combat, not only B. anthracis, but a wide range other infectious diseases.

### SUMMARY OF THE INVENTION

In a first embodiment, a method is provided for producing an antigenic composition comprising contacting an antigenic polypeptide and a second molecule comprising a reactive amino group with a sortase enzyme to produce an immunoconjugate. Administration of the immunoconjugate to a subject, can for instance, produce an immune response to the second molecule or an immune response to the second molecule and the antigenic polypeptide in the subject. For instance, the immune response to the second molecule can be enhanced relative to the immune response produced by administration of the second molecule alone. In certain aspects, the second molecule is non-antigenic when administered alone.

In a further embodiment an antigenic composition is provided comprising an isolated and essentially homogenous population (e.g., a population of molecules having the same structure) of antigenic polypeptide covalently linked to a bacterial capsule component wherein the covalent linkage comprises a peptide bond at the carboxyl-terminus of the antigenic polypeptide. For example, the covalent linkage between the antigenic polypeptide and the bacterial capsule component can consist of a peptide bond at the carboxyl-terminus of the antigenic polypeptide. In certain aspects, the carboxyl-terminus of the antigenic polypeptide, prior to linkage with the bacterial capsule component, comprises a recognition motif for a sortase enzyme, such as a sortase A, B, C or D recognition motif. Such an antigenic polypeptide can, in certain aspects, be engineered to comprise a sortase recognition motif that is not present in the native polypeptide. Thus, in certain aspects, the antigenic composition comprises the sequence LPXT, wherein X is any amino acid, at the carboxyl-terminus of the antigenic polypeptide (e.g., the polypeptide can comprise the sequence LPET). In some aspects, the antigenic polypeptide and the bacterial capsule component comprise the amino acid sequence LPXT (e.g., LPET) at the covalent linkage.

In still a further embodiment there is provided a method of producing an immune response in a subject comprising administering an antigenic composition or an immunoconjugate of the embodiments. For example, a method of the embodiments can be defined as a method of producing a protective or sterilizing immune response in a subject.

In yet a further embodiment there is provided a method of inducing a protective immune response in a subject comprising administering a composition comprising an antigenic portion of protective antigen (e.g., a portion of PA comprising the D4 domain) from B. anthracis conjugated to a poly-D-γ-glutamic acid (PDGA) capsule component, wherein administration of the composition provides resistance to B. anthracis-induced infection or mortality in the subject. In certain aspects, the PA and PDGA are chemically conjugated, such as by use of succinimidyl 3-(bromoacetamide) propionate (SBAP), 2-iminothiolane (ITL) or succinimidyl 4-formylbenzoate (SFB). In further aspects, PA and PDGA are enzymatically conjugated, for example, such that the carboxyl-terminus of the antigenic portion of PA is covalently attached to PDGA via a peptide bond. In certain cases, a method of the embodiments provides resistance to infection or mortality induced by B. anthracis that lacks a functional PagA gene product (e.g., a pagA mutant bacteria). In certain cases a method of the embodiments provides sterilizing immunity to B. anthracis, such that B. anthracis bacteria cannot be detected in the subject following bacterial challenge.

Antigenic polypeptides for use according the embodiments (e.g., for use in immunoconjugates) include, but are not limited, to antigenic polypeptides from a virus, bacteria, parasite or fungus or a polypeptide with adjuvant properties. For example, the antigenic polypeptide can be from a bacteria, such as a bacterial virulence factor. Such bacterial virulence factors include, without limitation, a virulence factor from Staphylococcus aureus (e.g., alpha hemolysin, a non-toxigenic form of alpha hemolysin, coagulase, von Willebrand factor binding protein, protein A, a non-toxigenic form of protein A, clumping factor A, clumping factor B, IsdA, IsdB, IsdH, FhuD2, EsxA or EsxB); Bordetella pertussis (such as pertussis toxin); Vibrio cholera (such as cholera toxin subunit A or B); Corynebacterium diphtheria (such as diphtheria toxin); Clostridium tetani (such as tetanus toxin); Pseudomonas aeruginosa (such as exotoxin A); Streptococcus pneumonia (such as pneumolysin); Streptococcus pyogenes (such as the C5a peptidase, M protein or T protein); Streptococcus agalactiae (such as, Rib, alpha-C, beta-C or BipA); Neisseria meningitides (such as a N. meningitides outer membrane protein); or H. influenza (such as H. influenzae-derived protein D). In certain aspects, the antigenic polypeptide is an antigen that, in vivo, may be anchored to or displayed on the cell wall of a Gram-positive bacteria. In certain aspects, the antigenic polypeptide is a sortase A, sortase B, sortase C, or sortase D substrate. In certain specific aspects, the antigenic polypeptide is from B. anthracis, such a B. anthracis protective antigen (PA) or an immunogenic fragment thereof. For example, the immunogenic fragment of PA can comprise the PA D4 domain (e.g., a portion of the protein corresponding to the amino acids encoded by a nucleic acid isolatable using PCR with primers having the sequence of SEQ ID NOs: 5 and 6).

In certain embodiments, a second molecule comprising a reactive amino group is used to produce an immunoconjugate. For example, the second molecule can be a peptide, a hapten, a peptidoglycan, a bacterial capsule component or a carbohydrate with a reactive amino group. The second molecule can, for instance, be a bacterial capsule component such as a capsular polysaccharide from Streptococcus pneumoniae, Neisseria meningitides, Staphylococcus aureus, Hemophilus influenza or Streptococcus agalactiae. In some aspects, the bacterial capsule component comprises a peptide chain such as a poly-γ-glutamic acid or the poly-D-γ-glutamic acid capsule of B. anthracis. In still further aspects, bacterial capsule component for use according to the embodiments can be chemically modified to add a reactive amino group.

In further aspects of the embodiments an antigenic polypeptide and a second molecule (e.g., a bacterial capsule component) of an immunoconjugate can be from the same or different organisms. For example, the antigenic polypeptide and the second molecule can both be from B. anthracis, such as PA and PDGA. Even in this situation, the immunoconjugate may be described as heterologous in the context of a linker because the PA and PDGA are not attached to one another in nature that way.

A sortase enzyme for use according to the embodiments can, in certain aspects, be an isolated sortase enzyme. For example the sortase enzyme can be a purified or recombinant sortase enzyme. Thus, in a further aspect, there is provided an immunoconjugate produced by the methods of the embodiments. Certain aspects of the embodiments concern sortase enzymes, such as a sortase A, sortase B, sortase C or sortase D enzyme. Sortase enzymes can be from a bacterial source, including but not limited to, sortase enzymes from Staphylococcus aureus, Bacillus anthracis or Bacillus cereus. For example, the sortase enzyme can be a sortase A from Staphylococcus aureus or Bacillus anthracis. Certain sortase enzymes and method for using the same are described in U.S. Pat. No. 7,238,489, incorporated herein by reference in its entirety.

In some embodiments, there is an immunogenic composition comprising a bacterial capsular component conjugated to a polypeptide via a linker comprising at least four amino acids of a sortase binding site, wherein the bacterial capsular component is immunogenic. In certain embodiments, the polypeptide is an antigenic polypeptide. In other embodiments, the polypeptide is a non-antigenic polypeptide. In further embodiments, the antigenic polypeptide comprises an antigenic amino acid sequence from a microbe. Some aspects concern a bacterial capsular component that is a bacterial polysaccharide. It is contemplated that the linker may be conjugated to the bacterial capsule component. In some cases, the conjugation occurred through transpeptidation.

Other embodiments concern a composition comprising an immunoconjugate comprising: a) a truncated anthrax protective antigen (PA) that comprises a D4 domain, wherein the PA is conjugated to a resulting sortase binding site, wherein the resulting sortase binding site has been previously cleaved by sortase; and, b) a bacterial capsular polysaccharide component. In some embodiments, the bacterial capsular polysaccharide component is a peptidoglycan. The peptidoglycan is PDGA in some embodiments.

Other embodiments of the invention are discussed throughout this application. Any embodiment discussed with respect to one aspect of the invention applies to other aspects of the invention as well and vice versa. The embodiments in the Example section are understood to be embodiments of the invention that are applicable to all aspects of the invention.

The terms “inhibiting,” “reducing,” or “prevention,” or any variation of these terms, when used in the claims and/or the specification includes any measurable decrease or complete inhibition to achieve a desired result.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed herein can be implemented with respect to any method or composition of the invention, and vice versa. Furthermore, compositions and kits of the invention can be used to achieve methods of the invention.

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.” It is also contemplated that anything listed using the term “or” may also be specifically excluded.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A-E: Protective antigen (pagA) deficient Bacillus anthracis Ames are not attenuated in mice. (FIG. 1A) Deletion of the pXO1-encoded pagA gene of B. anthracis Sterne via allelic replacement with the kanamycin (kan) resistance cassette. (FIG. 1B) Growth of wild-type and pagA mutant B. anthracis Ames in LB broth was monitored as the absorbance at 600 nm light. (FIG. 1C) B. anthracis wild-type (Ames) and pagA mutant strains were grown in the presence of carbon dioxide and stained with India ink to reveal the poly-D-γ-glutamic acid (PDGA) capsule. (FIG. 1D) Immunoblotting of B. anthracis culture supernatants with rabbit antibodies specific for Bs1A, EA1 and protective antigen (PA). (FIG. 1E) Wild-type B. anthracis (Ames), pagA and capD mutant spores were injected into the peritoneal cavity of C57Bl/6 mice and animal morbidity and mortality monitored. Statistical significance was examined with the log-rank test (* indicates P<0.01). Data are representative of two independent determinations.

FIG. 2A-C: Protective antigen (pagA) deficient Bacillus anthracis Ames are attenuated in the guinea pig model of anthrax disease. (FIG. 2A) Wild-type B. anthracis (Ames), pagA and capD mutant spores were injected into inguinal fold of guinea pigs and animal morbidity and mortality monitored. (FIG. 2B) Bacterial replication at the site of infection (SI) or dissemination into lung, liver and spleen was enumerated by plating homogenized tissues on agar media and incubation for colony formation. (FIG. 2C) Tissue from the site of infection (SI) of guinea pigs challenged with B. anthracis Ames or the pagA mutant strain as well as lung or spleen tissues were fixed, thin-sectioned, stained with hematoxylin-eosin and light microscopy images captured. Tissues from mock infected animals are included as a control. Statistical significance was examined with the unpaired student's t-test (* indicates P<0.01, **P<0.001, ***P<0.0001). Data are representative of two independent determinations.

FIG. 3A-C: AVA immunized guinea pigs are not protected against anthrax challenge with pagA mutant spores. (FIG. 3A) Guinea pigs (n=10) were immunized with a prime-two booster schedule with either AVA, PA adsorbed to Alhydrogel or mock (PBS/Alhydrogel) control in 14 day intervals. Animals were challenged by subcutaneous inoculation with B. anthracis Ames spores. (FIG. 3B) Immunized guinea pigs were challenged with pagA mutant spores. (FIG. 3C) Bacterial replication at the site of infection (SI) or dissemination into lung, liver and spleen was enumerated by plating homogenized guinea pig tissues on agar for colony formation. Statistical significance was examined with the unpaired student's t-test (* indicates P<0.01, **P<0.001).

FIG. 4A-C: Sortase conjugation generates the PDGA-D4 vaccine. (FIG. 4A) The sortase reaction scheme shows that capsule peptide is ligated onto PA-D4 LPETG sorting sequence through the attachment of N-terminal pentaglycine. (FIG. 4B) Sortase conjugation of PDGA to D4 (PDGA-D4) results in a mobility shift on 15% Coomassie-stained SDS-PAGE. (FIG. 4C) Using a prime-two booster schedule, guinea pigs were immunized with either D4 or PDGA-D4 adsorbed to Alhydrogel and serum IgG analyzed for immune reactivity to either D4 or PDGA antigen.

FIG. 5A-D: PDGA-D4 protects guinea pigs from wild-type as well as pagA mutant B. anthracis spore challenge. (FIG. 5A) Guinea pigs (n=7) were immunized with a prime-two booster schedule with either PDGA-D4 or D4 adsorbed to Alhydrogel or mock (PBS/Alhydrogel) control. Animals were challenged by subcutaneous inoculation with B. anthracis Ames spores. (FIG. 5B) Immunized guinea pigs (n=7) were challenged with pagA mutant spores. All guinea pigs were monitored for 14 days for disease and survival. The log-rank test was used to determine significance between PDGA-D4 and PBS mock immunized guinea pigs for wild-type B. anthracis AMES challenge (*P<0.01). For pagA challenge, PDGA-D4 offers significant protection when compared to D4 alone (*P<0.01). (FIG. 5C, D) Bacterial replication at the site of infection (SI) or dissemination into lung, liver and spleen from wild-type (FIG. 5C) or pagA (FIG. 5D) infected animals was enumerated by plating homogenized guinea pig tissues on agar for colony formation. After the 14 days of animal monitoring, all the survival guinea pigs were killed and tissues removed. No bacteria were detected in tissue homogenates from wild-type animas who survived the challenge (*P<0.01).

FIG. 6A-B: Histopathology of AVA or PA vaccinated guinea pigs following challenge with B. anthracis spores. (FIG. 6A) Guinea pigs (n=10) were immunized with a prime-two booster schedule with either AVA or PA adsorbed to Alhydrogel or a mock (PBS/Alhydrogel) control and challenged by subcutaneous inoculation with B. anthracis Ames (wild-type) or pagA mutant spores. Moribund animals were killed and the inguinal site of infection removed during necropsy. Tissues were fixed, thin-sectioned, stained with hematoxylin-eosin and light microscopy images captured. Tissues from mock infected animals are included as a control. (FIG. 6B) AVA or PA immunized guinea pigs (n=10) were challenged with wild-type or pagA mutant spores and thin sectioned hematoxylin-eosin stained lung tissues analyzed for histopathology.

FIG. 7: Spleen pathology of AVA or PA vaccinated guinea pigs challenged with wild-type or pagA mutant B. anthracis spores. Guinea pigs (n=10) were immunized with a prime-two booster schedule with either AVA or PA adsorbed to Alhydrogel or a mock (PBS/Alhydrogel) control and challenged by subcutaneous inoculation with B. anthracis Ames (wild-type) or pagA mutant spores. Moribund animals were killed and the spleen removed during necropsy. Spleen tissues were fixed, thin-sectioned, stained with hematoxylin-eosin and light microscopy images captured.

FIG. 8: Serum IgG immune response to anthrax vaccines. Guinea pigs (n=10) were immunized with a prime-two booster schedule in 14 day intervals using either AVA, PA, D4 or PDGA-D4 adsorbed to Alhydrogel or a mock (PBS/Alhydrogel) control. PA-specific serum IgG titers of animals were analyzed on day 36 of the immunization schedule.

FIG. 9A-E. Production and composition of recombinant PDGA. (A) Schematic representation of cap operon used for expression in E. coli. (B) High-molecular weight PDGA isolated from E. coli carrying operon shown in (A) and B. anthracis Ames. Samples were separated by agarose gel electrophoresis and stained with methylene blue (recombinant PDGA, left lanes; Ames PDGA, right lanes). (C) Schematic overview of derivatization reaction of D- and L-glutamate using Marfey's reagent. (D) HPLC chromatograms of derivatizied D- and L-form standards of glutamate were recorded at 350 nm. (E) HPLC chromatograms (350 nm) of processed PDGA samples. Relative amounts of D-form (fraction 21) and L-form (fraction 18) are indicated in percent.

FIG. 10A-B. (A) Nucleotide sequence pagA gene (gene pXO1-110 Sterne). Primers used to amplify D4 are underlined—D4 nt sequence is in red. (B) PagA Protective antigen full-length precursor. P13423[625-764], Protective antigen, Bacillus anthracis.

FIG. 11. capD mutant variant of cap operon of Bacillus anthracis (pXO2); nucleotides 1 to 4873 (SEQ ID NO:9).

### DETAILED DESCRIPTION OF THE INVENTION

The AVA vaccine has been subjected to preclinical efficacy trials in mice, rats, guinea pigs and non-human primates challenged with wild-type B. anthracis spores. However, B. anthracis Ames variants lacking the pXO1-encoded pagA gene remained virulent following either subcutaneous or respiratory spore challenge of mice. These studies suggest that AVA immunization or PA-specific antibodies are unlikely to protect mice against challenge with pagA mutant spores. Thus, new vaccine compositions are needed to provide protection against such strains.

Studies detailed here demonstrate that by conjugating antigenic portions of PA to PDGA a robust immunogen can be produced. Administration of the conjugates to guinea pigs elicited a robust antibody response. More importantly, the conjugate protected the animals from both virulent B. anthracis Ames variants and pagA mutant strains, that were not protected by PA vaccines alone. Moreover, the response to these immunoconjugates was sufficient to provide sterilizing immunity to the animals. Thus, PA-PDGA conjugates offer a significant advance relative to previously available B. anthracis immunogens in both overall efficacy and spectrum of immunity (i.e., effective immunity to pagA mutant strains).

The studies here likewise show the efficacy of new method for antigenic conjugation. Specifically, conjugation of a polypeptide and a second molecule, such as a bacterial capsule component, having a reactive amine can be accomplished by treatment with a sortase enzyme. The resulting conjugate comprises the antigenic polypeptide and a second molecule in a direct 1:1 ratio and the covalent linkage is at the same position relative to the two components (i.e., the carboxyl-terminus of the antigen polypeptide) in all conjugates. Thus, the antigenic polypeptide can effectively display the second molecule and facilitate a robust immune response to the molecule. The immune response to the antigenic polypeptide can also be enhanced in the immunoconjugate relative to the isolated antigenic polypeptide. Unlike chemical conjugation, all immunoconjugates produced by the method described herein are homogenous in their structure and thus offer compositions with a high and reproducible specific activity. Thus, the new conjugation procedures can be applied to a wide range of antigens to provide improved and highly active vaccine compositions.

I. Pharmaceutical Compositions and Methods

In some embodiments, pharmaceutical compositions are administered to a subject. Different aspects involve administering an effective amount of a composition to a subject. In some embodiments of the present invention, a composition comprising an immunoconjugate may be administered to the subject or patient to protect against or treat infection. Additionally, such compounds can be administered in combination with an adjuvant or an antibacterial therapy. In an embodiment, the adjuvant can comprise an aluminum salt adjuvant, such as aluminum hydroxide. Such compositions will generally be dissolved or dispersed in a pharmaceutically acceptable carrier or aqueous medium.

The active immunogens of the present invention can be formulated for parenteral administration, e.g., formulated for injection via the intravenous, intramuscular, subcutaneous, or even intraperitoneal routes. Typically, such compositions can be prepared as injectables, either as liquid solutions or suspensions; solid forms suitable for use to prepare solutions or suspensions upon the addition of a liquid prior to injection can also be prepared; and, the preparations can also be emulsified. In addition to the compounds formulated for parenteral administration, other pharmaceutically acceptable forms include, e.g., aerosolizable, inhalable, or instillable formulations; tablets or other solids for oral administration; time release capsules; creams; lotions; mouthwashes; and the like. The preparation of such formulations will be known to those of skill in the art in light of the present disclosure.

In certain embodiments, an immunoconjugate contains more than one compound against which an immune response is desired or beneficial or is associated with a protective or therapeutic immune response. Examples of these are described herein such as with an antigenic compound and a peptidoglycan found in the capsule of a capsular microbe. In some embodiments, however, the peptidoglycan component is connected to a polypeptide through a sortase binding site, but the polypeptide is not an antigenic polypeptide against which an immune response significantly contributes (i.e., is statistically significant and detectable) to the immunoconjugates' therapeutic effect. In certain embodiments, the second molecule generates an immune response that is the basis for a protective or therapeutic effect. In these embodiments, there is a peptide or polypeptide component that is attached to the second molecule, but the peptide or polypeptide is non-antigenic as described herein.

Antigenic or non-antigenic peptides or polypeptides may be 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 6000, 7000, 8000, 9000, 10000 or more amino acids in length, or any range derivable therein. In some embodiments, an antigenic polypeptide or peptide has a sequence that is at least or at most 75, 80, 85, 90, 91, 92, 93, 94, 95, 96, 97, 98, 99, or 100 percent identity or homology to the sequence of a particular antigenic polypeptide, such as can be found in GenBank. In particular embodiments, the antigenic polypeptide is a virulence factor from Staphylococcus aureus (e.g., alpha hemolysin, a non-toxigenic form of alpha hemolysin, coagulase, von Willebrand factor binding protein, protein A, a non-toxigenic form of protein A, clumping factor A, clumping factor B, IsdA, IsdB, IsdH, FhuD2, EsxA or EsxB); Bordetella pertussis (such as pertussis toxin); or is a protein from Vibrio cholera (such as cholera toxin subunit A or B); Corynebacterium diphtheria (such as diphtheria toxin), from Clostridium tetani (such as tetanus toxin); Pseudomonas aeruginosa (such as exotoxin A), from Streptococcus pneumonia (such as pneumolysin), from Streptococcus pyogenes (such as the C5a peptidase, M protein or T protein), from Streptococcus agalactiae (such as, Rib, alpha-C, beta-C or BipA), from Neisseria meningitides (such as a N. meningitides outer membrane protein), or from H. influenza (such as H. influenzae-derived protein D).

In specific embodiments, the antigenic polypeptide is protective antigen (PA) from anthrax. In some embodiments, the polypeptide comprises 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760. 761, 762, 763, or 764 amino acids from SEQ ID NO:10, which is the full-length sequence of a protective antigen protein from anthrax (MKKRKVLIPLMALSTILVSSTGNLEVIQAEVKQENRLLNESESSSQGLLGYYFSDLNFQ APMVVTSSTTGDLSIPSSELENIPSENQYFQSAIWSGFIKVKKSDEYTFATSADNHVTMW VDDQEVINKASNSNKIRLEKGRLYQIKIQYQRENPTEKGLDFKLYWTDSQNKKEVISSD NLQLPELKQKSSNSRKKRSTSAGPTVPDRDNDGIPDSLEVEGYTVDVKNKRTFLSPWISN IHEKKGLTKYKSSPEKWSTASDPYSDFEKVTGRIDKNVSPEARHPLVAAYPIVHVDMENI ILSKNEDQSTQNTDSQTRTISKNTSTSRTHTSEVHGNAEVHASFFDIGGSVSAGFSNSNSS TVAIDHSLSLAGERTWAETMGLNTADTARLNANIRYVNTGTAPIYNVLPTTSLVLGKNQ TLATIKAKENQLSQILAPNNYYPSKNLAPIALNAQDDFSSTPITMNYNQFLELEKTKQLR LDTDQVYGNIATYNFENGRVRVDTGSNWSEVLPQIQETTARIIFNGKDLNLVERRIAAV NPSDPLETTKPDMTLKEALKIAFGFNEPNGNLQYQGKDITEFDFNFDQQTSQNIKNQLAE LNATNIYTVLDKIKLNAKMNILIRDKRFHYDRNNIAVGADESVVKEAHREVINSSTEGLL LNIDKDIRKILSGYIVEIEDTEGLKEVINDRYDMLNISSLRQDGKTFIDFKKYNDKLPLYIS NPNYKVNVYAVTKENTIINPSENGDTSTNGIKKILIFSKKGYEIG). The signal peptide is amino acids 1-29. The full length protective antigen (without signal peptide) is amino acids 30-764. Protective antigen 20 (PA-20) is amino acids 30-196. Protective antigen 63 (PA-63) is amino acids 197-764. Embodiments include these different polypeptides. In further embodiments, a polypeptide is any of the lengths above but is at least or at most 80, 81, 82, 83, 84, 85, 86, 87, 88, 89, 90, 91, 92, 93, 94, 95, 96, 97, 98, 99, or 100% identical or homologous to the a polypeptide of that length from SEQ ID NO:10. In specific embodiments, a polypeptide that is included in an immunoconjugate is a truncated version of PA-63, which means it has fewer than amino acids 197-764 of SEQ ID NO:10. In additional embodiments, a polypeptide contains domain 4 but is lacking 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, or 700 residues (or any range derivable therein) from amino acids 197-764 of SEQ ID NO:10. In further embodiments, a polypeptide further is at most or at least 80, 81, 82, 83, 84, 85, 86, 87, 88, 89, 90, 91, 92, 93, 94, 95, 96, 97, 98, 99, or 100% identical or homologous to amino acids 197-764 with respect to the truncated protein.

The D4 sequence in SEQ ID NO:10 is 149 aa in length and aligns to PA from position 625 to 764 which is the C-terminal part of the mature PA-63. Embodiment include a PA polypeptide that comprises an amino acid sequence that is at most or at least 80, 81, 82, 83, 84, 85, 86, 87, 88, 89, 90, 91, 92, 93, 94, 95, 96, 97, 98, 99, or 100% identical or homologous to amino acids 625 to 764 of SEQ ID NO:10.

Embodiments include a second molecule against which an immune response can be generated. In some embodiments, a detectable immune response may not be generated against the second molecule but for the immunoconjugation described herein. In other embodiments the second molecule is made at least or at most about 10, 20, 30, 40, 50, 60, 70, 80, 90, 100 percent or at least about or at most about 1.5, 2.0, 2.5, 3.0, 3.5, 4.0, 4.5, 5.0 fold more immunogenic than prior to the conjugation. In certain embodiments, the second molecule is one found in the capsule of a bacteria, such as a Gram negative bacteria. In particular cases, the bacteria includes but is not limited to Escherichia coli, Klebsiella pneumonia; Haemophilus influenza; Bacillus megaterium; Pseudomonas aeruginosa; or, Salmonella. Gram-positive bacteria that have a capsule are also included in compositions and methods described herein. In specific embodiments, the second molecule is a polysaccharide or a peptidoglycan or another entity discussed herein.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

As used herein, the term “pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio. The term “pharmaceutically acceptable carrier,” means a pharmaceutically acceptable material, composition or vehicle, such as a liquid or solid filler, diluent, excipient, solvent or encapsulating material, involved in carrying or transporting a chemical agent.

Some variation in dosage will necessarily occur depending on the condition of the subject. The person responsible for administration will, in any event, determine the appropriate dose for the individual subject. An effective amount of therapeutic or prophylactic composition is determined based on the intended goal. The term “unit dose” or “dosage” refers to physically discrete units suitable for use in a subject, each unit containing a predetermined quantity of the composition calculated to produce the desired responses discussed above in association with its administration, i.e., the appropriate route and regimen. The quantity to be administered, both according to number of treatments and unit dose, depends on the effects desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition.

Upon formulation, solutions will be administered in a manner compatible with the dosage formulation and in such amount as is therapeutically or prophylactically effective. The formulations are easily administered in a variety of dosage forms, such as the type of injectable solutions described above.

Typically, for a human adult (weighing approximately 70 kilograms), from about 0.1 mg to about 3000 mg (including all values and ranges there between), or from about 5 mg to about 1000 mg (including all values and ranges there between), or from about 10 mg to about 100 mg (including all values and ranges there between), of a compound are administered. It is understood that these dosage ranges are by way of example only, and that administration can be adjusted depending on the factors known to the skilled artisan.

In certain embodiments, a subject is administered about, at least about, or at most about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 410, 420, 425, 430, 440, 441, 450, 460, 470, 475, 480, 490, 500, 510, 520, 525, 530, 540, 550, 560, 570, 575, 580, 590, 600, 610, 620, 625, 630, 640, 650, 660, 670, 675, 680, 690, 700, 710, 720, 725, 730, 740, 750, 760, 770, 775, 780, 790, 800, 810, 820, 825, 830, 840, 850, 860, 870, 875, 880, 890, 900, 910, 920, 925, 930, 940, 950, 960, 970, 975, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 6000, 7000, 8000, 9000, 10000 milligrams (mg) or micrograms (mcg) or μg/kg or micrograms/kg/minute or mg/kg/min or micrograms/kg/hour or mg/kg/hour, or any range derivable therein.

A dose may be administered on an as needed basis or every 1, 2, 3, 4, 5, 6, 7, 8, 9, 10, 11, 12, 18, or 24 hours (or any range derivable therein) or 1, 2, 3, 4, 5, 6, 7, 8, 9, or times per day (or any range derivable therein). A dose may be first administered before or after signs of an infection are exhibited or felt by a patient or after a clinician evaluates the patient for an infection. In some embodiments, the patient is administered a first dose of a regimen 1, 2, 3, 4, 5, 6, 7, 8, 9, 10, 11, 12 hours (or any range derivable therein) or 1, 2, 3, 4, or 5 days after the patient experiences or exhibits signs or symptoms of an infection (or any range derivable therein). The patient may be treated for 1, 2, 3, 4, 5, 6, 7, 8, 9, 10 or more days (or any range derivable therein) or until symptoms of an infection have disappeared or been reduced or after 6, 12, 18, or 24 hours or 1, 2, 3, 4, or 5 days after symptoms of an infection have disappeared or been reduced.

### EXAMPLES

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. The present examples, along with the methods described herein are presently representative of certain embodiments, are provided as an example, and are not intended as limitations on the scope of the invention. Changes therein and other uses which are encompassed within the spirit of the invention as defined by the scope of the claims will occur to those skilled in the art.

### Example 1

### Virulence of pagA Mutant B. anthracis Injected into the Peritoneal Cavity of Mice

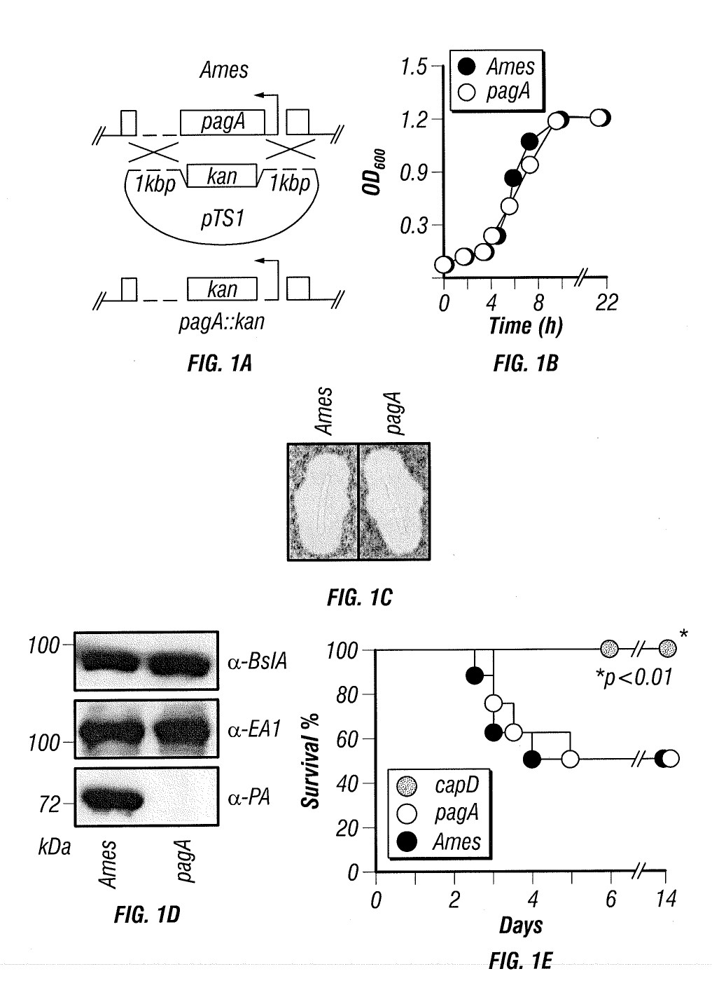

Using allelic replacement and phage transduction, the entire open reading frame of the pagA gene was deleted from the pXO1 virulence plasmid of B. anthracis Ames (FIG. 1A). The resulting pagA mutant strain formed spores and replicated in laboratory media at the same rate as wild-type bacilli (FIG. 1B). Growth in the presence of 5% CO₂ gas led to formation of encapsulated vegetative forms for both wild-type and pagA mutant bacilli (FIG. 1C). Cultures of vegetative bacilli grown in the presence of bicarbonate were centrifuged, thereby separating extracellular media and bacterial sediment. Proteins in the supernatant were precipitated with TCA and probed by immunoblotting (FIG. 1D). As expected, B. anthracis Ames secreted PA into the culture medium whereas the pagA mutant strain did not (FIG. 1D). As a control, the pagA mutant expressed the pXO1-encoded S-layer-associated protein BslA at a level similar to wild-type bacilli (FIG. 1D).

Next, studies were undertaken to determine whether or not the pagA mutant displayed the same level of virulence as the variant with the omega-kanamycin cassette replacement of pagA reported by Chand and colleagues. To test this, 300 spores derived from B. anthracis Ames or its pagA mutant were inoculated into the peritoneal cavity of C57BL/6 mice (FIG. 1E). Both wild-type and pagA mutant bacilli replicated in murine tissues and killed about half of the experimental animals. As a control, intraperitoneal injection of 300 spores derived from the B. anthracis Ames capD mutant did not cause lethal disease (FIG. 1E). Thus, it was concluded that the pagA deletion mutant of B. anthracis Ames is virulent in mice similar to the pagA omega-kanamycin cassette replacement variant.

### pagA Mutant Bacilli are Attenuated in the Guinea Pig Model of Anthrax Disease

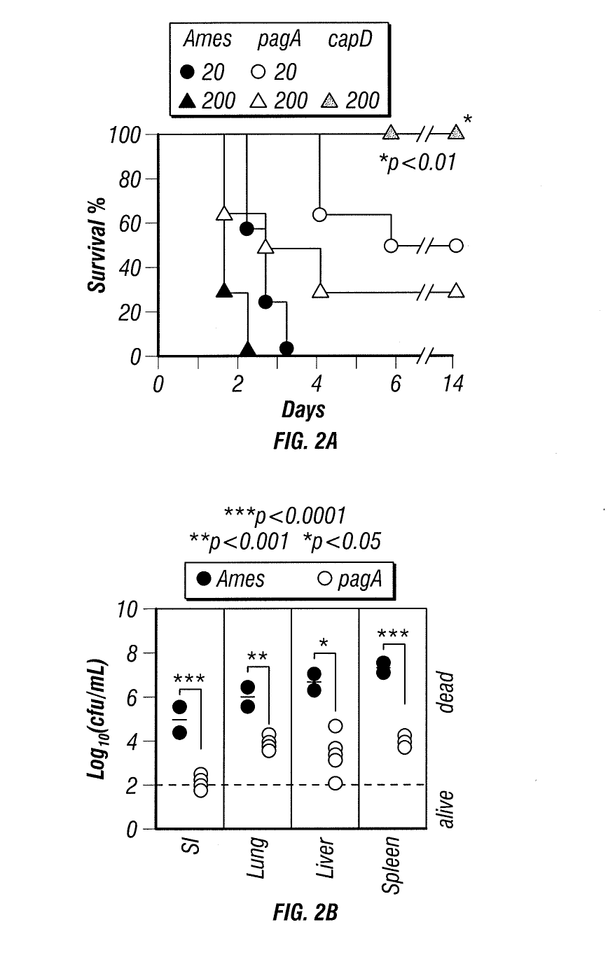

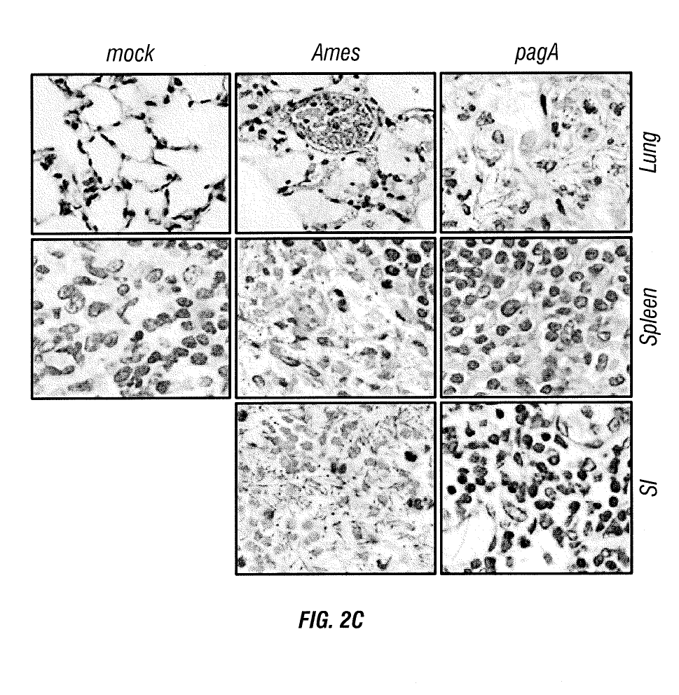

Cohorts of guinea pigs (n=10, 6-week old females) were infected with B. anthracis spores via subcutaneous injection into the inguinal fold. The survival of animals was monitored over the next fourteen days (FIG. 2A). All animals infected with spore preparations (20 or 200 CFU) of B. anthracis Ames succumbed to challenge within four days (FIG. 2A). By comparison, animals infected with pagA mutant spores displayed delayed time-to-death and increased survival (FIG. 2A). Thus, the pagA mutation attenuates B. anthracis Ames virulence in guinea pigs about 10 fold; by comparison, a capD mutation is known to reduce virulence 1,000 fold. To study the disease features of the pagA mutant, animals were subjected to necropsy and tissue from the site of infection (inguinal fold), liver, lung and spleen was homogenized, spread on agar plates and colony formation enumerated as a measure for bacterial load (FIG. 2B). Tissue samples were also fixed with formalin, embedded, thin-sectioned and stained with hematoxylin-eosin (FIG. 2C). Animals infected with wild-type B. anthracis Ames spores displayed massive replication of vegetative forms at the injection site (10⁵ CFU) as well as pathogen dissemination to lung, liver and spleen (FIG. 2B). Edema and hemorrhage were detected at the site of infection (FIG. 2C). Histopathology revealed B. anthracis vegetative forms with massive invasion of polymorphonuclear leukocytes and macrophages in lung and spleen tissues (FIG. 2C). By comparison, vegetative replication of the pagA mutant at the site of infection was reduced by about 3 log₁₀ CFU (P<0.0001) and did not elicit edema or hemorrhage (FIG. 2C). Dissemination of the pagA mutant to liver, lung and spleen tissues was also reduced by 2-3 log₁₀ CFU (FIG. 2B).

### AVA or PA Immunized Guinea Pigs are Susceptible to Infection with pagA Bacilli

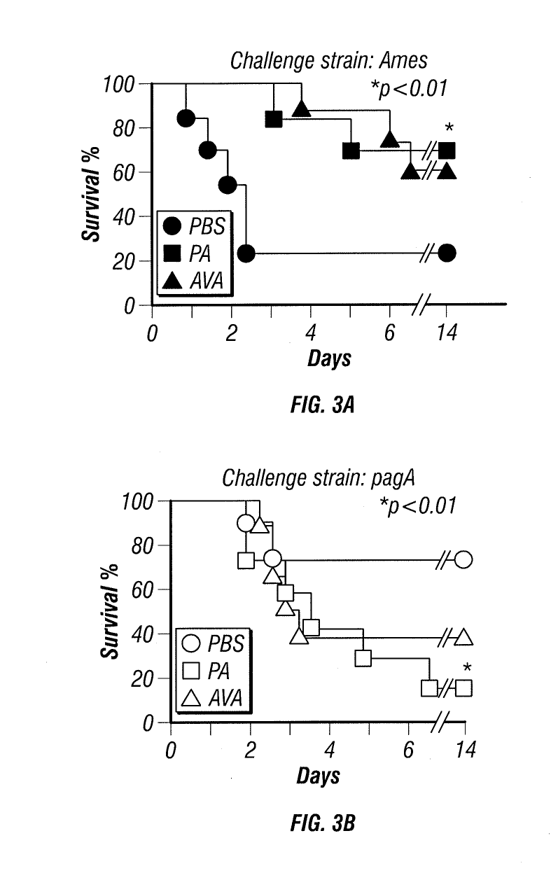

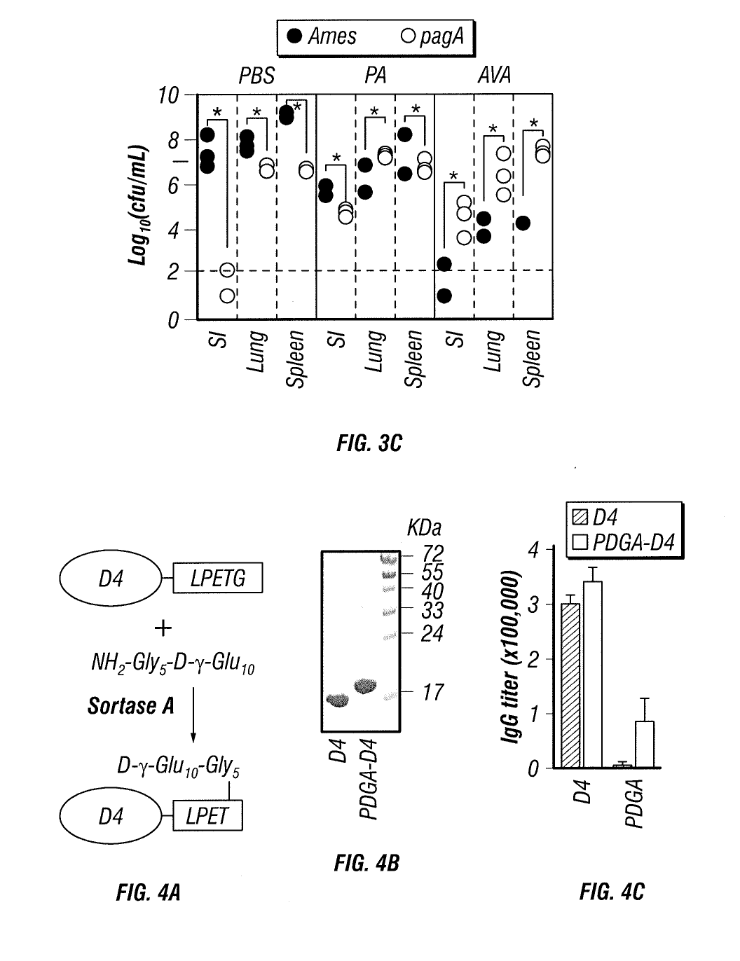

Cohorts of guinea-pigs (n=10) were immunized by intramuscular injection with three doses of 50 μg PA adsorbed to aluminum hydroxide or 250 μl AVA (Biothrax®) in fourteen day intervals. Two-weeks following the final immunization, animals were challenged with spores derived from B. anthracis Ames or its pagA mutant (FIG. 3). PA as well as AVA immunization of guinea pigs afforded protection from B. anthracis Ames challenge, as both vaccine protocols caused similar increases in animal survival and time-to-death over a control cohort of mock (PBS) immunized guinea pigs exposed to the same challenge dose (FIG. 3A). AVA immunization, and to a lesser degree vaccination with PA, reduced the bacterial load at the site of infection as well as in lung, liver and spleen tissues (FIG. 2C). Nevertheless, neither of the two vaccines elicited full protection from anthrax disease (FIG. 3AB). This is an expected result, as optimal protection is only achieved upon completion of a schedule of 5-6 AVA immunizations over a one-year period.

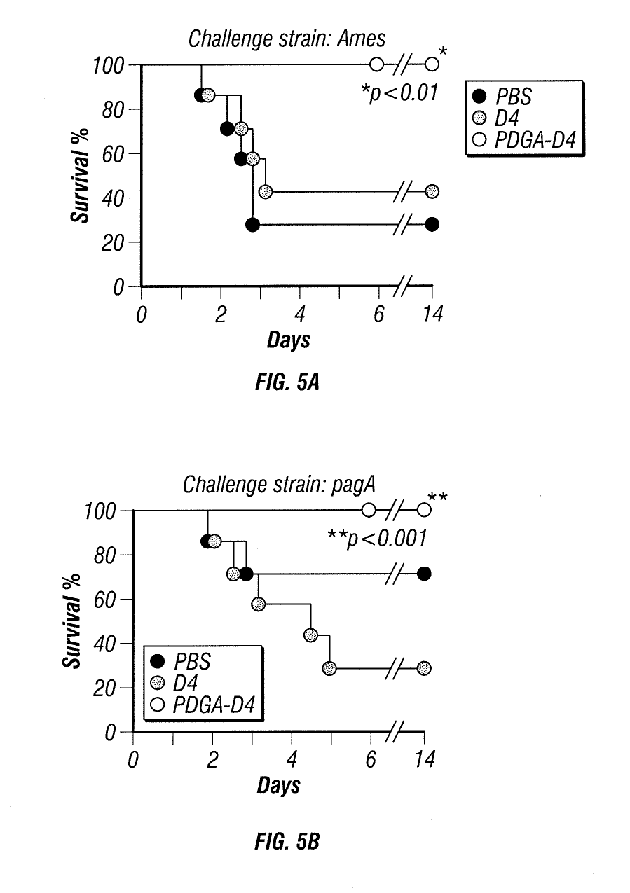

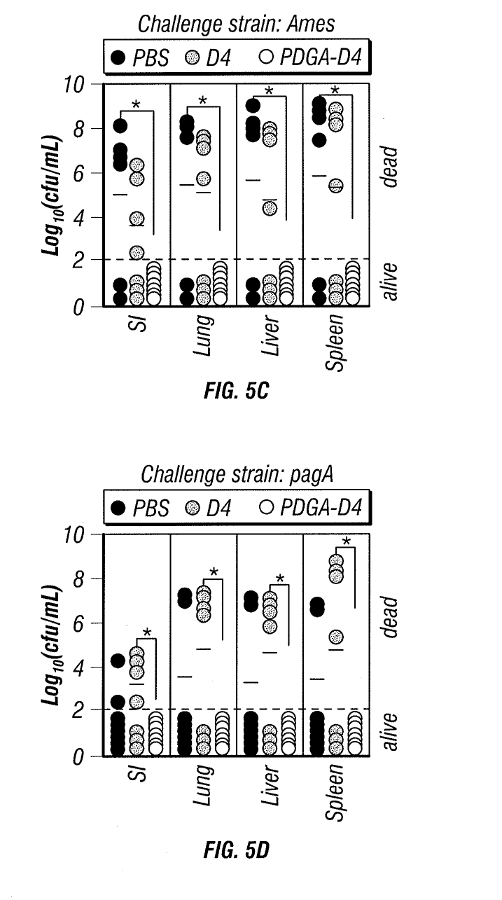

Surprisingly, AVA immunized animals succumbed at a faster rate to challenge with pagA mutant spores than animals of the mock-immunized control cohort (FIG. 3B). This phenomenon must be caused by immune responses to the protective antigen as guinea pigs immunized with purified PA (FIG. 3B) or the D4 domain of PA (FIG. 5B) exhibited similar hyper-sensitivity to challenge with spores derived from pagA mutant B. anthracis Ames. To test whether immunization with AVA or PA affected the ability of pagA mutants to replicate in host tissues, the bacterial load at the injection site and distant organ sites was examined. Compared to mock immunized animals, AVA vaccinated guinea pigs harbored a dramatically increased load of the pagA mutant at the site of infection (FIG. 3C).

### Pathological Features of Anthrax Disease Caused by Wild-Type and pagA Mutant Bacilli

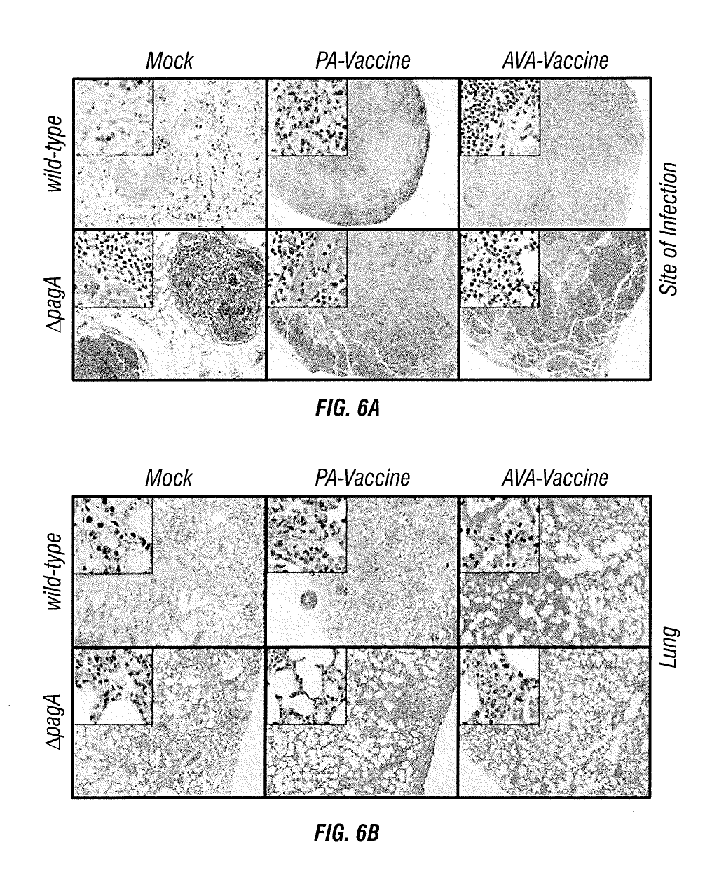

The studies shown in FIG. 3 suggest that AVA immunization enhanced the virulence attributes of the pagA mutant B. anthracis Ames strain. This conjecture is further supported by the histopathology of infected guinea pigs (FIG. 6). AVA and PA immunization ameliorate the hemorrhagic lesions, immune cell necrosis and vegetative replication associated with B. anthracis Ames spore inoculation at the site of infection (FIG. 6A). PA and AVA vaccinated animals established a granuloma at the site of infection, which appeared to restrict replication and dissemination of the germinated pathogen (FIG. 6A). In mock immunized animals, vegetative replication of the pagA mutant occurred within a similar granuloma (FIG. 6A). Site of infection lesions in AVA or PA immunized guinea pigs were marked by hemorrhagic zones and severe necrosis of immune cells (FIG. 6). Mock immunized guinea pigs infected with B. anthracis Ames spores developed severe interstitial pneumonia with a large burden of vegetative bacilli (FIG. 6B). Lung histopathology of the pagA mutant showed a large immune cell infiltrate and moderate replication of the pathogen (FIG. 6B). AVA or PA immunization limited pathogen replication within the lung: only sporadic interstitial immune cell infiltrates and occasional vegetative forms could be detected (FIG. 6B). When challenged with pagA mutant bacilli, lung interstitial space of AVA or PA-immunized guinea pigs was enlarged by infiltrates of healthy or necrotic immune cells and the vegetative forms of the pagA mutant strain (FIG. 6B).

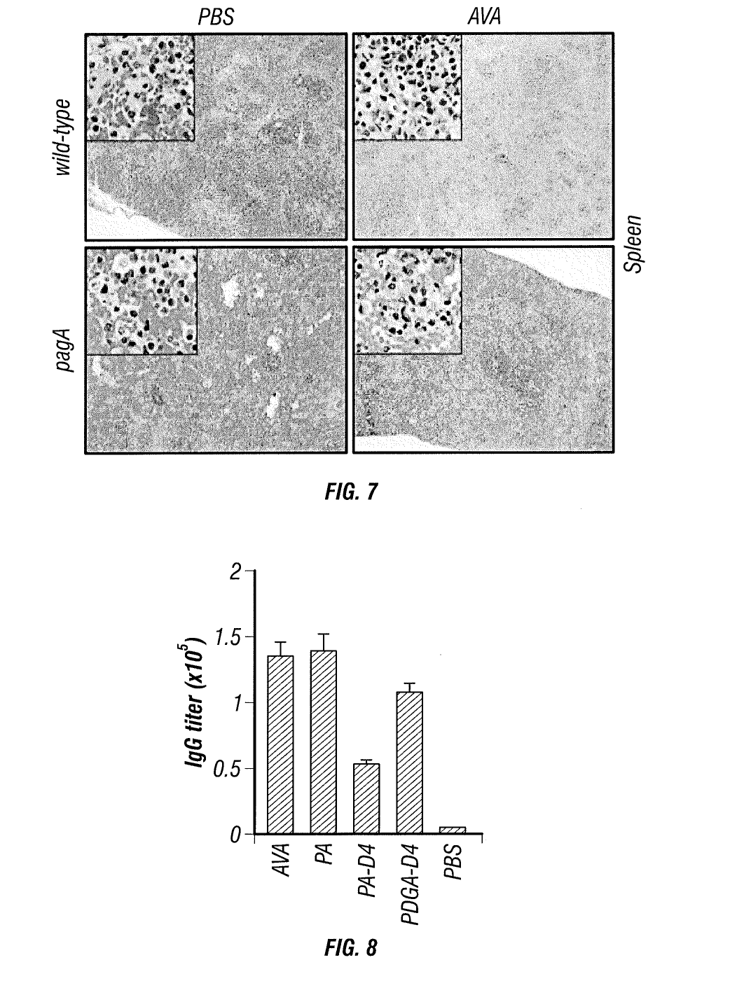

Virulent B. anthracis replicate to the highest numbers in the spleen of their infected hosts (FIG. 2). As expected, large numbers of B. anthracis Ames vegetative forms established microcolonies in spleen tissues of mock immunized animals (FIG. 7). Vegetative forms of pagA mutant were associated with moderate immune cell infiltrates without changing the overall architecture of the spleen (FIG. 7). Splenic tissues of AVA immunized guinea pigs did not reveal anthrax pathology when infected with B. anthracis Ames spores (FIG. 7). However, AVA-vaccinated animals challenged with pagA mutant spores displayed vegetative forms and immune cell infiltrates replacing the red and white pulp architecture of spleen tissues (FIG. 7).

### Sortase-Ligation Generates Conjugate Vaccines

Chemical cross-linking has been used to tether PDGA to PA carrier, thereby enabling MHC presentation and antibody responses to otherwise non-immunogenic capsular material. Nevertheless, the ability of such PDGA-PA conjugate vaccine to elicit protection against wild-type and pagA mutant B. anthracis Ames has not yet been explored. Sortase A, a transpeptidase that cleaves between the threonine (T) and glycine (G) of its LPXTG recognition motif, was therefore exploited for the development of conjugate vaccines (FIG. 4A). A glutathione S-transferase hybrid with the D4 receptor binding domain of PA, which is the target of neutralizing PA antibodies, was engineered to harbor a C-terminal LPXTG motif Purified GST-D4 was incubated with sortase A and NH₂-Gly₅-γ-D-Glu₁₀ nucleophile. The transpeptidation product, D4-LPXT-Gly₅-γ-D-Glu₁₀ (designated PDGA-D4), was cleaved off GST and purified (FIG. 4B). When injected into guinea pigs, Alhydrogel adsorbed PDGA-D4 elicited antibody responses against PDGA capsule and the D4 domain of PA (FIG. 4C and FIG. 8). As a control, non-conjugated D4 antigen did not elicit PDGA antibodies (FIG. 4C).

### PDGA-D4 Protects Guinea Pigs Against Wild-Type and pagA Mutant Bacilli

Guinea pigs were immunized with PDGA-D4 or D4 following the same schedule as AVA and challenged with B. anthracis Ames spores (FIG. 5A). In contrast to D4 immunized animals, which exhibited partial protection against B. anthracis Ames spore challenge, guinea pigs that have received PDGA-D4 were completely protected (FIG. 5A). At the end of the observation period, animals were euthanized and subjected to necropsy. Microbiological analysis of tissue homogenates from PDGA-D4 immunized guinea pigs failed to detect B. anthracis, suggesting that the conjugate vaccine induced sterilizing immunity (FIG. 5C). Importantly, PDGA-D4 immunization protected guinea pigs against challenge with the pagA mutant strain (FIG. 5BD). Compared to mock immunized animals, immunization with the D4 antigen reduced the survival of guinea pigs subsequently challenged with the pagA mutant strains (FIG. 5B).

### Example 2

### Materials and Methods

Bacillus anthracis Growth and Spore Preparations

B. anthracis cultures were grown overnight in Luria broth with or without 0.85% sodium bicarbonate at 37° C. and diluted in fresh medium at 37° C. Antibiotics were added to cultures for plasmid selection: 100 μg/ml ampicillin and 50 μg/ml kanamycin for Escherichia coli strains and 20 μg/ml kanamycin for B. anthracis strains. For spore preparation, vegetative cultures of B. anthracis Ames wild-type, pagA or capD mutants were sporulated in modified G medium [0.2% yeast extract, 0.0025% CaCl₂ dihydrate, 0.05% KH₂PO₄, 0.00976% MgSO₄ anhydrous, 0.005% MnCl₂.4H₂O, 0.00073% ZnSO₄.7H₂O, 0.00005% FeSO₄.7H₂O, 0.2% (NH₄)₂SO₄] until >99% sporulation was observed by light microscopy. Endospores were heat-treated at 68° C. for 1 h to kill vegetative cell. Spores were washed with sterile ddH₂O three times, suspended in sterile H₂O and stored frozen at −80° C. Endospore preparations were plated on LB agar to determine CFUs. Endospore preparations were examined by microscopy and found to be >99% purity with no observable vegetative cells or debris. For capsule production B. anthracis strains were grown in a capsule inducing medium [0.8% nutrient broth (pH 6.8), 0.3% yeast extract, 0.7% NaHCO₃, 10% horse serum, 25 mM HEPES-KOH, pH 7.5, 1.5% agar] overnight at 37° C. in 5% CO₂.

Bacillus anthracis Mutants and Plasmids

B. anthracis Sterne 34F2 pXO1 was used as a template for PCR amplification of two 1 kb DNA fragments flanking the pagA gene using the primers pagA1 (5′-TTTGGATCCGAGATGAAAATGGTAATATAGCGAATA-3′; SEQ ID NO:1) and pagA2 (5′-TTTCCCGGGATACGTTCTCCTTTTTGTATAAAATTAAA-3′; SEQ ID NO:2) (PCR 1) as well as pagA3 (5′-TTTCCCGGG GGTAATTCTAGGTGATTTTTAAATTATCT-3′; SEQ ID NO:3) and pagA 4 (5′-TTTGAATTCATGTGCCATTGTTTTTAAAAGTTC-3′; SEQ ID NO:4) (PCR2). PCR products 1 and 2 were restricted with BamH1/XmaI and XmaI/EcoRI, respectively, and ligated into pTS1 cut with BamH1/EcoR1. The recombinant plasmid, pJWK374A was cut with SmaI and ligated to the kanamycin resistance cassette flanked by SmaI1 sites to generate pJWK374B. Plasmid pJWK374B was transformed into E. coli strain K1077 (dam dcm), non-methylated DNA purified and electroporated into B. anthracis Sterne as previously described (Gaspar A H, et al. (2005)). Allelic replacement and selection for a kan resistant pagA mutant followed the protocol of Marraffini (Marraffini L A & Schneewind O (2006)). Nucleic acid sequences of wild-type and mutant allele were verified by DNA sequencing. The capD variant of B. anthracis Ames has been previously described in Richter G S, et al. (2009), which is incorporated herein by reference in its entirety). The B. anthracis Sterne pagA mutant allele was transduced into B. anthracis Ames strain using CP-51 phage. In brief, the B. anthracis pagA Sterne mutant was grown overnight at 30° C. in NBY supplemented with 0.5% glycerol broth and kanamycin 20 μg/ml, and then refreshed in NBY supplemented with 0.5% glycerol for 3-5 h at 37° C. Following infection of 100 μl of refreshed donor strain with 100 μl CP-51 WT phage stock, 4 ml of PA soft agar was added and the transduction mix was plated on NBY plates with 0.5% glycerol. Following 30-46 hours incubation at 30° C., the soft agar was scraped off into 5 ml PA broth. Following centrifugation, the supernatant was passed through 0.22 μm sterile filter.

Fractionation of B. anthracis Cultures

B. anthracis strains were grown overnight in LB with or without 0.8% sodium bicarbonate as indicated. Overnight cultures were diluted 1:100 in fresh medium and grown to an optical density of 3 at 600 nm (A600 3). Total proteins in the cell culture (Total) were obtained by precipitating 1 ml of the culture with 7.5% trichloroacetic acid (TCA). To assay for protein secretion in the medium, 3 ml of the culture was centrifuged for 5 min at 6,000×g. Proteins in 1 ml of supernatant (Sup) were precipitated with 7.5% trichloroacetic acid (TCA). All TCA precipitates were washed with ice-cold acetone, solubilized in 50 μl of 0.5 M Tris-HCl (pH 8.0)/4% SDS and heated at 90° C. for 10 min. Proteins were separated on SDS/PAGE and transferred to PVDF membrane for immunoblot analysis with appropriate rabbit polyclonal antibodies. Immunoreactive signals were revealed by using a secondary antibody coupled to horseradish peroxidase and chemiluminescence.

### Sortase Conjugation

The D4 domain sequence was amplified with the primers D4-1 (5′-tttggatcctttcattatgatagaaataacatagcagttg-3′; SEQ ID NO:5) and D4-2 (5′-tttgaattcttattcacccgtagccggaagagcttgagctcctatctcatagcatttttagaaa-3′; SEQ ID NO:6) primers using pXO1 template DNA. The PCR product was digested with BamHI and EcoRI, inserted into pGEX-2T (GE healthcare, USA) to generate pYT10, which was transformed into E. coli and transformants selected on Luria agar with ampicillin (100 μg/ml). An N-terminal truncated recombinant sortase A (SrtA_{ΔN}) was purified as described previously (Ton-That H, Liu G, Mazmanian S K, Faull K F, & Schneewind O (1999)). For GST-D4 purification, E. coli BL21 (DE3) (pYT10) was grown in LB broth at 37° C. to OD₆₀₀ 0.6 and induced with 1 mM IPTG for 3 hrs. Cells were sedimented by centrifugation, suspended in 30 mL buffer A [50 mM Tris-HCl (pH 7.5), 150 mM NaCl] and lysed in a French pressure cell at 14,000 psi. The extract was centrifuged at 29,000×g for 30 min, and the supernatant was applied to 1 ml of glutathione agarose, pre-equilibrated with buffer A. The column was washed with 40 ml of buffer A, and GST-D4 protein was eluted in 5 ml of buffer B [50 mM Tris-HCl (pH 7.5), 150 mM NaCl, 20 mM glutathione]. Excess glutathione was removed by dialysis against buffer A.

### PDGA Production and the Use of this PDGA in Synthesizing the Conjugate Vaccine

In B. anthracis, capsule biosynthesis and anchoring is encoded by the capBCADE operon on the virulence plasmid pXO2. The anthrax capsule filaments consist of poly-γ-D-glutamic acid (PDGA). CapBC catalyzes PDGA synthesis in the cytoplasm and the CapAE transmembrane complexes transport the polymers across the cell membrane. On the cell surface, CapD attaches the polymers to a peptidoglycan by covalently linking the γ-carboxyl groups of PDGA to the side chain amino groups of the meso-diaminopimelic acid (m-DAP) of the muropeptide.

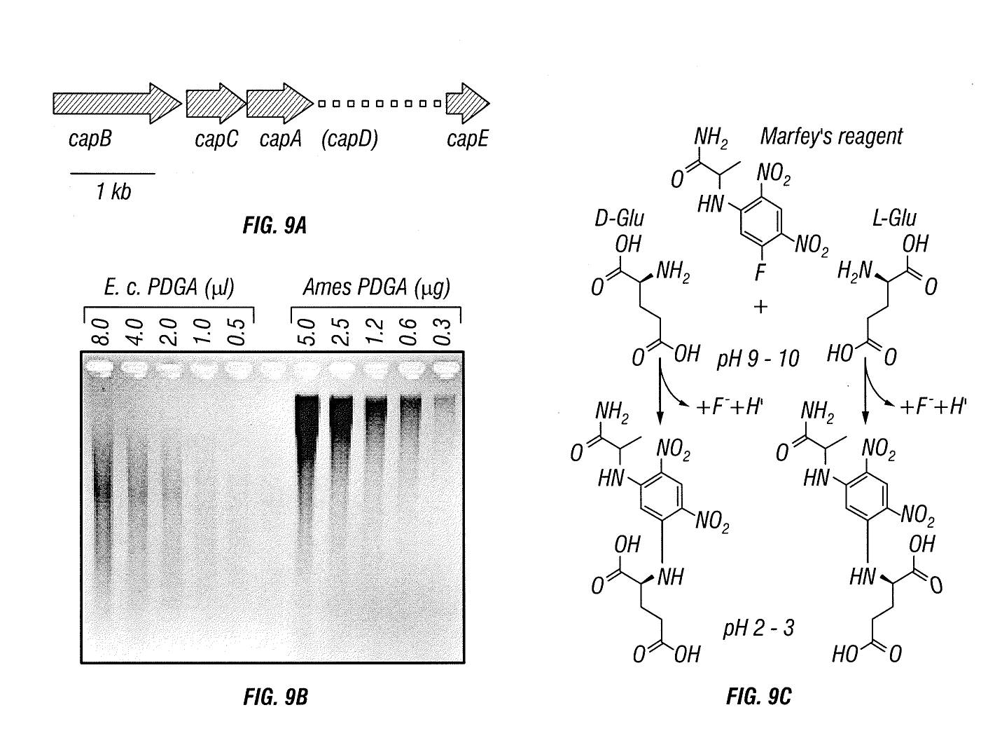

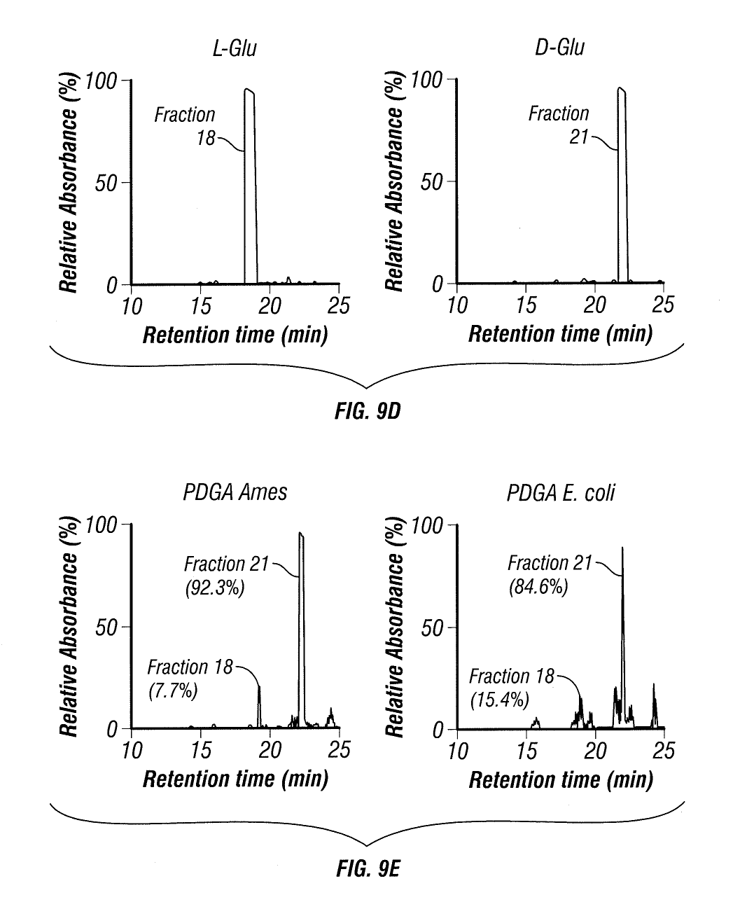

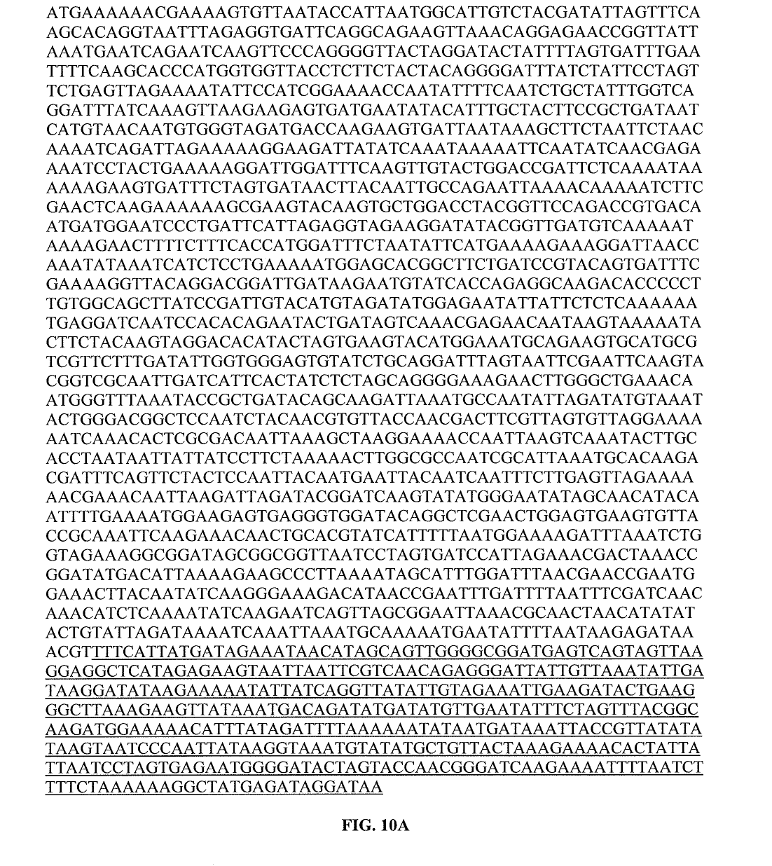

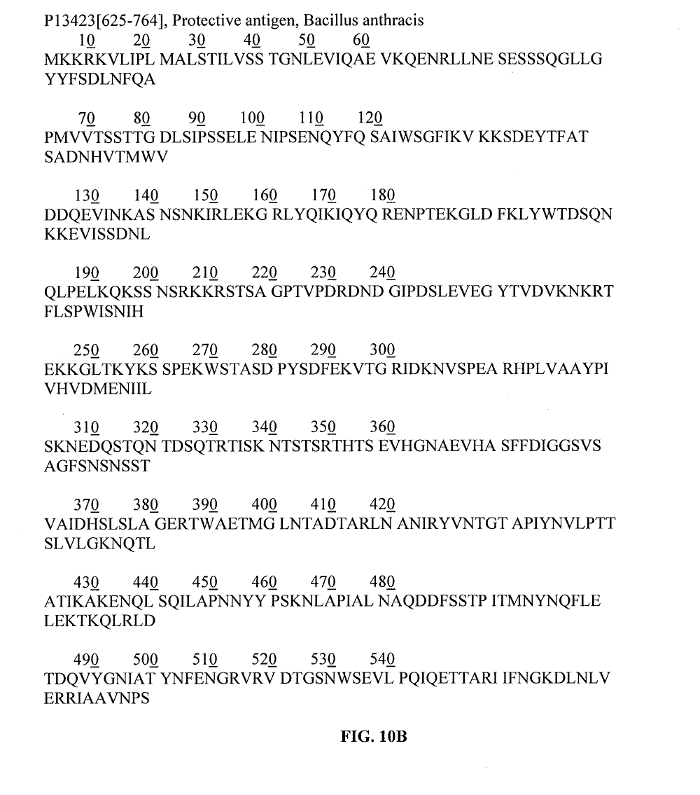

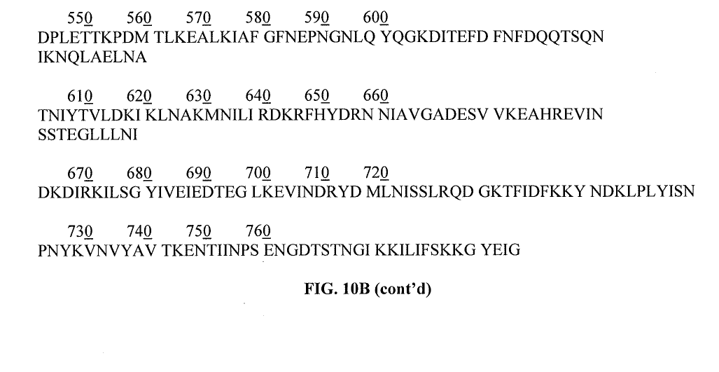

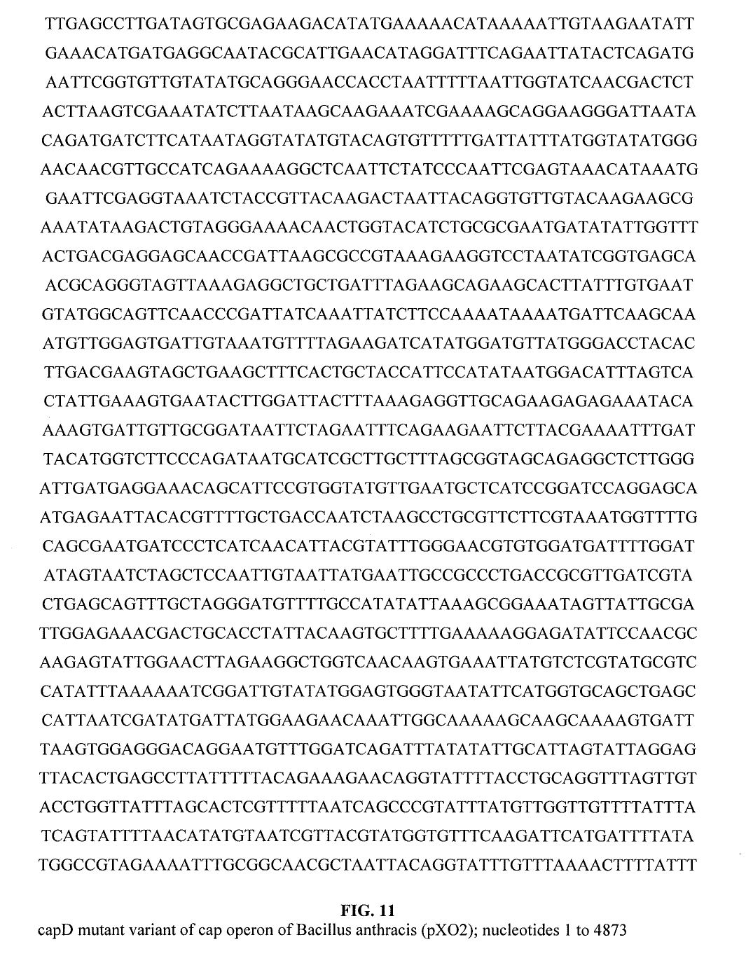

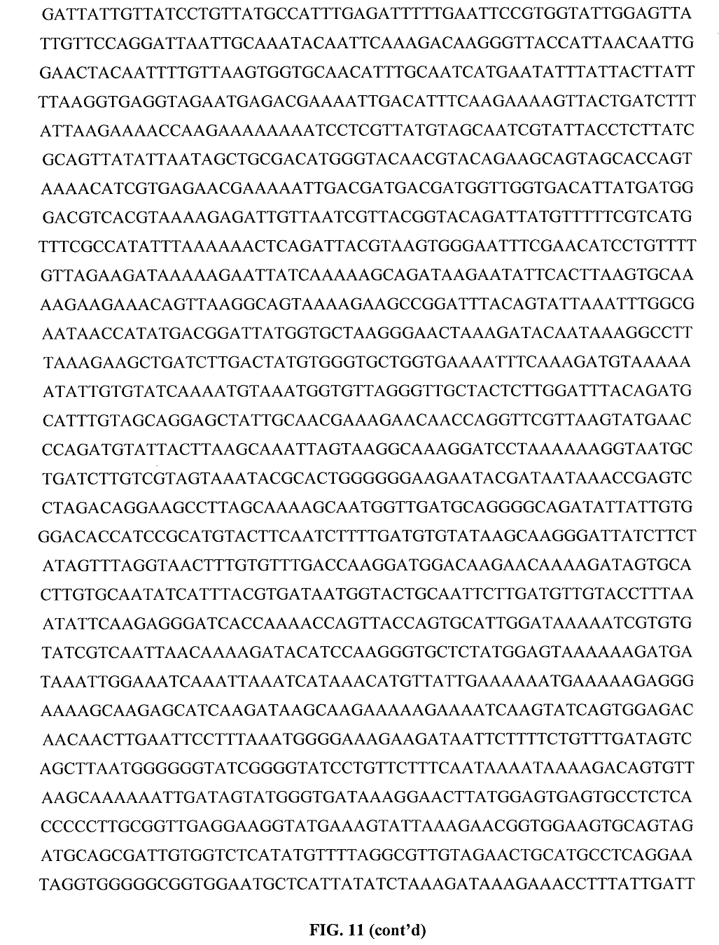

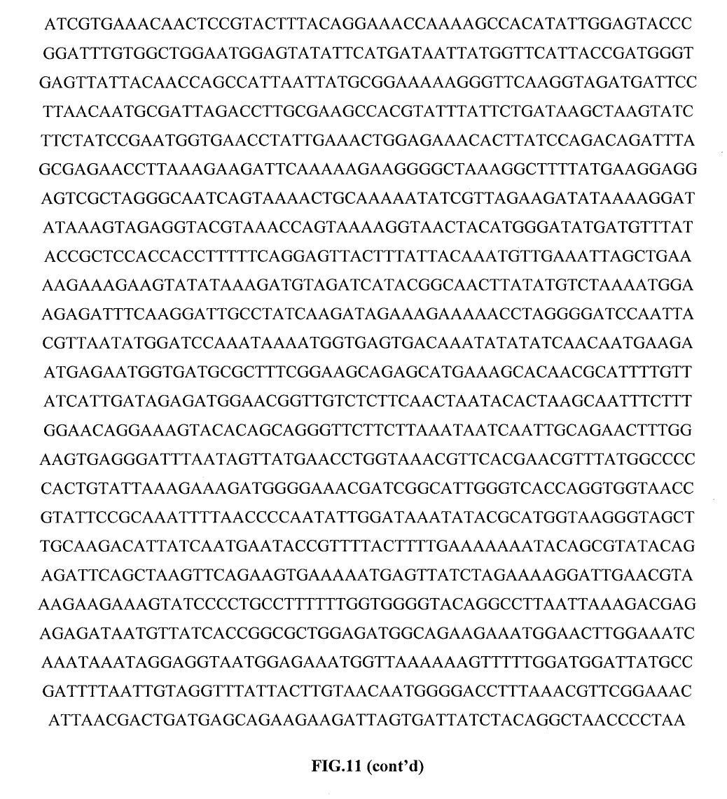

To produce recombinant PDGA, a genetically altered version of the cap operon was utilized. To generate an altered version of the cap operon, a total DNA preparation of B. anthracis Ames was used as template in a PCR to amplify the intact capBCADE coding region (FIG. 9A) flanked by an upstream KpnI site and a downstream EagI site for cloning into expression vector pLM5 with Kan^{R} gene for selection of transformants. The cap operon encoded by the complement strand of virulence plasmid pXO2 (GenBank accession number NC_{—}007323, which is hereby incorporated by reference) was amplified by a forward primer incorporating a KpnI restriction site (SEQ ID NO 7) (5′-TGTCGA GGTACC TTGAGCCTTGATAGTGCGAG-3) (sequence in bold represents pXO2 complement strand positions 57,020 to 57,001; flanking KpnI site introduced for cloning is underlined) and reverse primer incorporating an EagI restriction site (SEQ ID NO 8) (5′-CTAACA CGGCCG TTAGGGGTTAGCCTGTAGAT-3′) (sequence in bold represents pXO2 positions 52,147 to 52,166; flanking EagI site introduced for cloning is underlined). The 4.9-kb PCR product (corresponding to positions 52,147 to 57,020 on pXO2) was digested with the restrictases and inserted into the corresponding cloning sites on pLM5, Marraffini et al. 2006), downstream of the IPTG-inducible spat promoter. The resulting construct pCAP1 was propagated in E. coli DH5a and then transformed into E. coli K1077 (dam⁻ dcm⁻) to screen for PDGA-producing clones.

Minicultures of transformants were grown in selection medium (LB containing 50 μg×ml-1 kanamycin) supplemented with 1 mM IPTG for inducing the cap operon. After overnight incubation, culture supernatants were recovered by centrifugation and heat-treated for 20 min at 95° C. Cell pellets were immediately frozen at −80° C. For cell lysis, the pellets were thawed in 10 mM HEPES-KOH, pH 7.5, and treated with a sonicator until the lysates turned clear. Insoluble material was removed by centrifugation.

To precipitate PDGA from culture supernatants, 4 volumes of ethanol were added and samples were stored at −20° C. for 48 hours. Precipitates were recovered by centrifugation and dissolved in 10 mM HEPES-KOH, pH 7.5. Both supernatant and cell pellet samples were treated with 200 μg×ml⁻¹ proteinase K for 2 hours at 37° C. The enzyme was heat-inactivated (20 min at 95° C.) and insoluble material was removed by centrifugation (FIG. 9B). Partial digestion of high molecular weight PDGA with CapD is required to obtain a fragmented product with reactive amino groups that are needed for conjugation. Samples were tested for PDGA content by dot blot probed with an anti-capsule antibody. Plasmids from PDGA-positive clones were isolated and sequenced. Analysis revealed that the capsule-producing clone CAP1-5 (SEQ ID NO:9) carries a cap operon with a frame shift mutation in capD that inactivates the gene without a polar effect on the other genes. PDGA isolated from this strain has been analyzed for D-glutamic acid content and was used in immunoconjugation experiments.

To determine the D-glutamate content of recombinant PDGA, the purified product was hydrolyzed by incubation at 105° C. in pH 2 for 16 hours. This lyzed material was neutralized and incubated with Marfey's reagent (Sigma) that reacts with primary amines for UV detection (FIG. 9C). Derivatized D- and L-glutamates were separated by reversed-phase HPLC with a linear gradient of acetonitrile in ammonium formate-methanol buffer on a ODS column (particle size 5 μm; column size 250×4.6 mm; Thermo Scientific). Glutamate enantiomers differ in their retention time with the D=form eluting after the L=form (FIG. 9D). The HPLC protocol was optimized using commercial D- and L-glutamate preparations (Sigma) as standards. The analysis indicates that recombinant PDGA contains 85% D-form. Capsule isolated from fully virulent B. anthracis Ames has 92% D-glutamate (FIG. 9E).

The transpeptidation reaction was carried out in 0.5 ml 100 mM HEPES, 150 mM NaCl, 5 mM CaCl₂ (pH 7.5) with 0.2 mM GST-D4, 0.1 mM sortase and 1 mM nucleophile (NH₂-Gly₅-PDGA₁₀) for 12 hours at 37° C. Reaction products were subjected to glutathione agarose affinity chromatography. After washing with buffer A, GST tag was cleaved off with thrombin. Ion-exchange chromatography through MonoQ column (GE-healthcare, USA) was used to purify PDGA-D4 from D4. The reaction products were analyzed by analytical RP-HPLC and characterized with ESI-MS.

### Animal Models of Anthrax Infection

All animal experiments followed protocols that were reviewed, approved, and supervised by the Institutional Animal Care and Use Committee and the Select Agent Committee at the University of Chicago. Six week old, female C57BL/6 mice (Jackson Laboratory) were challenged by intraperitoneal injection of B. anthracis spore suspensions in 100 μl PBS. Aliquots of the spore inoculum were spread on agar plates to enumerate the challenge dose. Infected animals were monitored in 12 hour intervals for survival or a moribund state (inability to remain upright, weight loss or un-responsive to touch). Moribund animals were killed by inhalation of compressed CO₂ and removal of vital organs.

Six week old, female C57BL/6 mice (Jackson Laboratory) were challenged by intra-peritoneal injection of B. anthracis spore suspensions in 100 μl PBS. Aliquots of the spore inoculum were spread on agar plates to enumerate the challenge dose. Infected animals were monitored in 12 hour intervals for survival or a moribund state (inability to remain upright, weight loss, non-responsive to touch) for 14 days. Moribund animals were killed by inhalation of compressed CO₂ and cervical dislocation.

Female Hartley guinea pigs (250-350 g) were infected by subcutaneous injection of spores into the inguinal fold of the hind leg. Animals were observed for morbidity and mortality for 14 days. All animals were subjected to necropsy and their site of infection, spleen, liver and lungs removed. Organs were immediately fixed by submersion in 10% neutral-buffered formalin and embedded in paraffin. Samples were submitted to the University of Chicago Animal Pathology Core for serial 4-μm thin sections and staining with hematoxylin-eosin. Tissue samples were viewed by light microscopy. Organ samples isolated during necropsy were also homogenized in phosphate buffered saline, serially diluted, and plated on LB to enumerate bacterial load as CFU. Alternatively, samples were fixed with neutral buffered formalin and stained with India ink to visualize the capsule of bacilli.

Groups (n=10) of female Hartley guinea-pigs (250-280 g) were immunized by intramuscular injection into the hind leg with 0.2 ml of 50 μg of PA (List Biological Laboratories, Inc.), D4 or PDGA-D4 absorbed to 25% aluminum hydroxide (Alhydrogel) on days 1, 14, 28. As a control, 250 μl AVA (Biothrax®) was administered at the same time. Blood was collected on day 35 to measure serum antibody titers before challenge. Levels of guinea pig serum immunoglobulin G (IgG) reactive with specific antigens were determined by a custom enzyme-linked immunosorbent assay (ELISA). Briefly, serum samples representative of the immunization groups were aliquoted on microtiter plates pre-coated with purified PA, D4 (5 μg/ml) or PDGA-D4 (20 μg/ml). Binding of serum antibody was detected with secondary antibodies against specific immunoglobulin (anti-guinea pig).

### Statistical Analysis

Data were processed using GraphPad PRISM® 5.0 software to generate graphs and for statistical analyses. Statistical analysis of serum antibody levels was performed in pairwise comparison using the unpaired two-tailed Student's t-test. Bacterial load data were analyzed for statistical significance with the unpaired two-tailed Student's t-test. Comparisons of animal survival between two groups were evaluated with the log-rank test.

### REFERENCES

The following references, to the extent that they provide exemplary procedural or other details supplementary to those set forth herein, are specifically incorporated herein by reference.
1. Koch R (1876) Die Ätiologie der Milzbrand-Krankheit, begründet auf die Entwicklungsgeschichte des Bacillus anthracis. Beiträge zur Biologie der Pflanzen 2:277-310.
2. Preisz H (1909) Experimentelle Studien über Virulenz, Empfänglichkeit and Immunität beim Milzbrand. Zeitschrift für Immunitäts-Forschung 5:341-452.
3. Bruckner V, Kovacs J, & Denes G (1953) Structure of poly-D-glutamic acid isolated from capsulated strains of B. anthracis. Nature 172:508.
4. Smith H, Keppie J, & Stanley J L (1955) The chemical basis of the virulence of. Bacillus anthracis. V. The specific toxin produced by Bacillus anthracis in vivo. Br. J. Exp. Pathol. 36:460-472.
5. Collier R J & Young J A (2003) Anthrax toxin. Annu. Rev. Cell Dev. Biol. 19:45-70.
6. Bradley K A, Mogridge J, Mourez M, Collier R J, & Young J A (2001) Identification of the cellular receptor for anthrax toxin. Nature 414:225-229.
7. Duesbery N S, et al. (1998) Proteolytic inactivation of Map-kinase-kinase by anthrax lethal factor. Science 280:734-737.
8. Leppla S H (1982) Anthrax toxin edema factor: a bacterial adenylate cyclase that increases cyclin AMP concentrations in eukaryotic cells. Proc. Natl. Acad. Sci. USA 79:3162-3166.
9. Green B D, Battisti L, Koehler T M, Thorne C B, & Ivins B E (1985) Demonstration of a capsule plasmid in Bacillus anthracis. Infect. Immun. 49:291-297.
10. Ivins B E, et al. (1986) Immunization studies with attenuated strains of Bacillus anthracis. Infect. Immun. 52:454-458.
11. Pasteur L (1881) Le vaccin du charbon. Comptes Rendus Hebdomadaires des Scéances de l'Académie des Sciences 92:666-668.
12. Sterne M (1988) Anthrax vaccines. J. Am. Vet. Med. Assoc. 192:141.
13. Dixon T C, Meselson M, Guillemin J, & Hanna P C (1999) Anthrax. N. Engl. J. Med. 341:815-826.
14. Meselson M (1999) The challenge of biological and chemical weapons. Bull. World Health Organ. 77:102-103.
15. Grabenstein J D (2008) Vaccines: countering anthrax: vaccines and immunoglobulins. Clin. Infect. Dis. 46:129-136.
16. Singer D E, et al. (2008) Serum IgG antibody response to the protective antigen (PA) of Bacillus anthracis induced by anthrax vaccine adsorbed (AVA) among U.S. military personnel. Vaccine 26:869-873.
17. Schneerson R, et al. (2003) Poly(gamma-D-glutamic acid) protein conjugates induce IgG antibodies in mice to the capsule of Bacillus anthracis: a potential addition to the anthrax vaccine. Proc. Nat. Acad. Sci. USA 100:8945-8950.
18. Kubler-Kielb J, et al. (2006) Additional conjugation methods and immunogenicity of Bacillus anthracis poly-gamma-D-glutamic acid-protein conjugates. Infect. Immun. 74:4744-4749.
19. Little S F, et al. (1996) Characterization of lethal factor binding and cell receptor binding domains of protective antigen of Bacillus anthracis using monoclonal antibodies. Microbiology 142:707-715.
20. Chen Z, et al. (2011) Pre- and postexposure protection against virulent anthrax infection in mice by humanized monoclonal antibodies to Bacillus anthracis capsule. Proc. Natl. Acad. Sci. USA 108:739-744.
21. Chand H S, et al. (2009) Discriminating virulence mechanisms among Bacillus anthracis strains by using a murine subcutaneous infection model. Infect. Immun. 77:429-435.
22. Drysdale M, et al. (2005) Capsule synthesis by Bacillus anthracis is required for dissemination in murine inhalation anthrax. EMBO J. 24:221-227.
23. Twenhafel NA (2010) Pathology of inhalational anthrax animal models. Vet. Pathol. 47:819-830.
24. Richter G S, et al. (2009) Capsule anchoring in Bacillus anthracis occurs by a transpeptidation mechanism that is inhibited by capsidin. Mol. Microbiol. 71:404-420.
25. Ton-That H, Liu G, Mazmanian S K, Faull K F, & Schneewind O (1999) Purification and characterization of sortase, the transpeptidase that cleaves surface proteins of Staphylococcus aureus at the LPXTG motif. Proc. Natl. Acad. Sci. USA 96:12424-12429.
26. Brossier F, Levy M, Landier A, Lafaye P, & Mock M (2004) Functional analysis of Bacillus anthracis protective antigen by using neutralizing monoclonal antibodies. Infect. Immun. 72:6313-6317.
27. Marraffini L A, DeDent A C, & Schneewind O (2006) Sortases and the art of anchoring proteins to the envelopes of gram-positive bacteria. Microbiol. Mol. Biol. Rev. 70:192-221.
28. Cornelius C A, Quenee L E, Elli D, Ciletti N A, & Schneewind O (2009) Yersinia pestis IS1541 transposition provides for escape from plague immunity. Infect. Immun. 77:1807-1816.
29. Drysdale M, Bourgogne A, & Koehler T M (2005) Transcriptional analysis of the Bacillus anthracis capsule regulators. J. Bacteriol. 187:5108-5114.
30. Kim H U & Goepfert J M (1974) A sporulation medium for Bacillus anthracis. J. Appl. Bacteriol. 37:265-267.
31. Gaspar A H, et al. (2005) Bacillus anthracis sortase A (SrtA) anchors LPXTG motif-containing surface proteins to the cell wall envelope. J. Bacteriol. 187:4646-4655.
32. Marraffini L A & Schneewind O (2006) Targeting proteins to the cell wall of sporulating Bacillus anthracis. Mol. Microbiol. 62:1402-1417.

1. A method for producing an antigenic composition comprising contacting an antigenic polypeptide and a second molecule comprising a reactive amino group with an isolated sortase enzyme to produce an immunoconjugate, wherein administration of the immunoconjugate to a subject produces an immune response to the second molecule.

2. The method of claim 1, wherein the second molecule is a peptide, a hapten, a peptidoglycan or a carbohydrate with a reactive amino group.

3. The method of claim 1, wherein the second molecule is a bacterial capsule component.

4. The method of claim 1, wherein the antigenic polypeptide and the second molecule are from the same organism.

5. The method of claim 4, wherein the antigenic polypeptide and the second molecule are both from B. anthracis.

6.-11. (canceled)

12. The method of claim 1, wherein the antigenic polypeptide is the protective antigen (PA) from B. anthracis or an antigenic fragment thereof.

13. The method of claim 12, wherein antigenic polypeptide is a fragment of PA comprising the D4 domain.

14. The method of claim 3, wherein the bacterial capsule component is a capsular polysaccharide from Streptococcus pneumoniae, Neisseria meningitides, Staphylococcus aureus, Hemophilus influenza or Streptococcus agalactiae.

15. The method of claim 3, wherein the bacterial capsule component comprises poly-γ-glutamic acid.

16. The method of claim 15, wherein the bacterial capsule component is the poly-D-γ-glutamic acid capsule of B. anthracis.

17.-22. (canceled)

23. An antigenic composition comprising an isolated and essentially homogenous population of antigenic polypeptide covalently linked to a bacterial capsule component wherein the covalent linkage comprises a peptide bond at the carboxyl-terminus of the antigenic polypeptide.

24. The antigenic composition of claim 23, wherein the antigenic polypeptide and the covalently linked bacterial capsule component are present in a 1:1 ratio.

25.-28. (canceled)

29. The antigenic composition of claim 23, wherein the covalent linkage between the antigenic polypeptide and the bacterial capsule component consists of a peptide bond at the carboxyl-terminus of the antigenic polypeptide.

30. The antigenic composition of claim 23, wherein the antigenic polypeptide and the bacterial capsule component are from the same organism.

31. The antigenic composition of claim 30, wherein the antigenic polypeptide and the bacterial capsule component are both from B. anthracis.

32. The antigenic composition of claim 23, wherein the antigenic polypeptide is the protective antigen (PA) from B. anthracis or an antigenic fragment thereof.

33. The antigenic composition of claim 32, wherein antigenic polypeptide is a fragment of PA comprising the D4 domain.

34. The antigenic composition of claim 23, wherein the bacterial capsule component comprises poly-γ-glutamic acid.

35. The antigenic composition of claim 34, wherein the bacterial capsule component is the poly-D-γ-glutamic acid capsule of B. anthracis.

36. (canceled)

37. A method of inducing a protective immune response in a subject comprising administering a composition comprising an antigenic portion of protective antigen (PA) from B. anthracis conjugated to a poly-D-γ-glutamic acid capsule component, wherein administration of the composition provides resistance to B. anthracis-induced mortality in the subject.

38.-60. (canceled)
